(19) United States
(12) Patent Application Publication
(10) Pub. No.: US 2026/0273207 A1
(43) Pub. Date: Sep. 17, 2026

(54) NEBULIZER FOR A FLUID CONTAINED IN A CONTAINER

(71) Applicant: NEUTEC INHALER ILAC SANAYI VE TICARET ANONIM SIRKETI, Istanbul (TR)

(72) Inventor: Mahmut BILGIC, Istanbul (TR)

(21) Appl. No.: 19/471,637

(22) PCT Filed: Jun. 2, 2023
(86) PCT No.: PCT/IB2023/055693

§ 371 Date: Oct. 1, 2025

(30) Foreign Application Priority Data
Apr. 6, 2023 (WO) PCT/IB2023/053508

(51) Int. Cl. A61M 15/00

(52) U.S. Cl. CPC A61M 15/009; A61M 15/0026

(57) ABSTRACT

The invention relates to a nebulizer (1) for a fluid (3) located in a container (2), having a first housing part (G1), which has a mouthpiece (5), and a second housing part (G2), which forms a receiving room (10) for the container (2), the first and second housing parts (G1 and G2) being rotatable relative to one another about a longitudinal housing axis (x) in preparation for an atomising process, the first housing part (G1) further having a helical bevel surface (31), for acting on a retaining part (16) for the container (2), which retaining part (16) is axially dis-placeable relative to the first and second housing parts (G1 and G2) when connected rotationally fixed to the second housing part (G2). In order to advantageously further design A nebulizer of the type in question, it is proposed that a third housing part (G3) is provided, which surrounds the first housing part (G1) on the outside and is connected to the first housing part (G1) in a rotationally fixed but exchangeable manner.

### FIELD OF TECHNOLOGY

The invention relates to a nebulizer for a fluid located in a container, having a first housing part, which has a mouthpiece, and a second housing part, which forms a receiving room for the container, the first and second housing parts being rotatable relative to one another about a longitudinal housing axis in preparation for an atomising process, the first housing part further having a helical bevel surface for acting on a retaining part for the container, which retaining part is axially displaceable relative to the first and second housing parts when rotationally fixedly connected to the second housing part.

### STATE OF THE ART

Nebulizers of the type in question are known, for example, from WO 2007/022898 A2 (U.S. Pat. No. 7,823,584 B2) and further, for example, from WO 2012/160052 A1 (U.S. Pat. No. 9,827,384 B2), WO 1997/012687 A1 (U.S. Pat. No. 5,964,446 A) or also from WO 2017/080895 A1 (US 2017/0128681 A1).

The container thereby forms, whereby this description also concerns possible parts and modes of operation of the nebulizer according to the invention, a reservoir for an atomising fluid, whereby such a container can be inserted, if necessary replaceably, into the receiving space of the nebulizer prior to initial use of the nebulizer. The container may have a rigid outer shell, for example made of a metal material, such as aluminium, and an inner bag in which the fluid is stored. Preferably, the fluid is stored in the container without pressure, i.e. without propellant gas or the like.

Specifically, as a result of the rotational displacement of the housing parts relative to each other, the container within the housing is lowered, together with a hollow piston located within it. During the lowering, a spring acting on the container is tensioned. At the same time, fluid flows into a pressure chamber located outside the container. This can be achieved by a negative pressure caused in the pressure chamber. Through a release actuation, the container together with the hollow piston located therein can be released for a pressure movement upwards. The fluid in the pressure chamber is pressurised and then ejected as a spray. For this purpose, the hollow piston can preferably be provided with a non-return valve at its end associated with the pressure chamber so that it can act as a piston.

Thus, through the preferred rotational displacement of the first and the second housing part relative to each other, a predetermined amount of fluid (dosing amount) is brought into a chamber, from which the divided amount of fluid is expelled voluntarily by user intervention and atomised into an aerosol for inhalation by the user. The expulsion of the fluid dose takes place as a result of pressurisation, which pressure is built up in the course of the relative rotation of the first and second housing parts with respect to each other as a result of the tension of a spring. The fluid to be expelled can be subjected to a pressure of, for example, about 1 to 40 MPa, further for example 10 to 25 MPa, whereby with each stroke a fluid delivery of about 5 to 50 μl, further for example about 10 to 20 μl, further for example about 15 μl can take place. During atomisation into an aerosol, droplets with an aerodynamic diameter of, for example, up to 20 μm, preferably approximately between 3 and 10 μm, can be created.

Furthermore, nebulizers known from the above-mentioned publications are designed in such a way that they can no longer be used after a predefined maximum number of atomisation processes has been carried out, i.e. if necessary—at least theoretically—after the container has been completely emptied, and are disposed of. When the predetermined maximum number of atomisation processes is reached, a means comes into effect that prevents further use of the nebulizer. Also known from the above-mentioned publications are nebulizers in which the container can be exchanged for a new, unused and thus completely filled container when the maximum number of atomisation processes is reached.

### SUMMARY OF THE INVENTION

With regard to the aforementioned prior art, the invention has the task of advantageously further designing a nebulizer of the type in question.

According to a first idea of the invention, a possible solution to the problem is given in a nebulizer, in which it is intended that a third housing part is provided, which surrounds the first housing part on the outside and is connected to the first housing part in a rotationally fixed but exchangeable manner.

The two-part design of the upper housing part as a whole achieved in this way advantageously offers the possibility of a different design of the outer third housing part, which is essentially visible to the user and can be handled, while the design of the inner first housing part preferably remains the same.

This first housing part can, as is also preferred, be designed to be essentially solely functional. Thus, the first housing part preferably has the mouthpiece, and more preferably also a possible pressure chamber and/or a nozzle body. In addition, a trigger mechanism for triggering an atomisation impact can also be arranged or provided on or in the inner first housing part.

Together with the second, lower housing part, a nebulizer torso consisting of the inner housing parts can result, which essentially has all the components and assemblies relevant for the function of the nebulizer, so further also the receiving room for the container or also the retaining part. Such a nebulizer torso can also be subjected to a function test, for example, without the arrangement of the outer, third housing part.

The outer third housing part, which is connected to the first housing part in a rotationally fixed manner and cannot be displaced relative to the first housing part even along the longitudinal axis of the housing during normal use of the nebulizer, can be designed alone, for example with regard to the outer design of the nebulizer and/or for example with regard to the feel and/or for example with regard to the operability of the nebulizer as a whole.

The two upper housing parts, inner, first housing part and outer, third housing part, form an upper housing part pair which is easy for the user to operate and which preferably cannot be separated into the two housing parts again, at least not without tools and/or destruction, during normal use of the Nebulizer. This design, however, gives the advantage in view of producing the nebulizer that concerning the third housing part a change use of a possible modified third housing part can be made, without making a change of other housing parts necessary. Parts, which are immediately combined or related, as e.g. the release button and/or the housing cap can be changed together therewith.

A combination between the third housing part and the first housing part can be made on different ways. First, there can be a lodging, further a welding and also a press combination. The combination is at least chosen such, that a user cannot detach the parts by hand. Further, also a bonding is possible.

Above and below, the first housing part is often also described as a fixed and/or as an inner, upper housing part, the second housing part is often described as a relative to this first housing part rotatable housing part and/or as an inner, lower housing part, the third housing part is often described as a housing part, connected the first housing part non-rotatably, and/or as an outer, upper housing part, and a possible fourth housing part is often described as a housing part, connected the second housing part non-rotatably, and/or as an outer, lower housing part.

Further features of the invention are explained below, also in the description of the figures, often in their preferred assignment to the subject-matter of claim 1 or to features of further claims. However, they may also be of significance in an assignment only to individual features of claim 1 or of the respective further claim, or in each case independently.

Thus, according to a possible embodiment, it can be provided that the third housing part is combined, e.g. latched, to the first housing part—with reference to a usual use orientation of the nebulizer in which the longitudinal housing axis is oriented substantially vertically—above a lower end of the helical bevel surface. A latching plane extending transversely to the longitudinal axis of the housing can be established, which is spaced in the axial direction from the end of the helical bevel surface by a dimension which can correspond to approximately 0.2 to 0.4 times the axial extension of the third housing part, not taking into account a cap which may be provided as a component of the third housing part.

The first housing part can form a circumferential shoulder for a stop, e.g. a latching stop, when the third housing part is placed on the first housing part. Preferably, the third housing part is pushed onto the first housing part essentially in the axial direction, further preferably starting from the end of the first housing part that has the mouthpiece. In addition, the e.g. established latching plane, established in the area of the shoulder, can be essentially assigned to a transition area of the mouthpiece into the first housing part in other respects.

In an advantageous further development, it is provided that a fourth housing part is snap-fitted to the second housing part. With this fourth housing part, together with the second housing part, an almost free external design of the entire nebulizer can be achieved with an essentially constant nebulizer torso. The fourth housing part can also be variably designed, for example, with regard to the feel and/or, for example, the operability of the nebulizer. The snap-in mounting of the fourth housing part on the second housing part preferably also provides a rotationally fixed connection between these housing parts, so that the overall result for the user is a pair of lower housing parts that is easy to operate.

The latch between the fourth housing part and the second housing part may not be able to be released by the user, at least not without tools and/or without being destroyed. Alternatively, however, the user can also be given the option of releasing the latch by deliberate intervention, for example to replace an emptied container with a new container, in particular in the case of a nebulizer for multiple use.

The fourth housing part can be opposite the third housing part in a parting plane extending transversely to the longitudinal axis of the housing, in which parting plane the third and fourth housing parts can be rotated relative to each other. The parting plane of the outer enclosure parts can be visible to the user from the outside. In addition, this parting plane may well be axially offset from a parting plane between the inner, first and second housing parts.

In a further embodiment, a cover cap can be attached to the third, upper housing part. For example, a snap-in fastening can be provided in this regard, which may only occur in the closed position covering the mouthpiece.

In a further embodiment, the cover cap can be connected to the third housing part via a hinge joint, whereby the cover cap can be held captive on the third housing part in an advantageous manner. A geometric pivot axis may, as preferred, extend transversely to the longitudinal housing axis. In the cap-off position, which corresponds to the use position of the nebulizer, the cover cap can also serve as an abutment when gripping the upper housing part and rotating the lower housing part relative to the upper housing part.

Like the third housing part, the fourth housing part can also be a one-piece manufactured part, in particular a one-piece injection-moulded (plastic) part. Alternatively, there is the possibility of a design and/or functional extension, in particular for the fourth housing part, by manufacturing it from two partial housing parts that can be arranged in one another in a pot-like manner. The two partial housing parts can thus be designed differently, for example in terms of colour and/or also in terms of the material used, whereby it may also be possible to achieve a different feel between the housing parts.

The two partial housing parts can be connected to each other, for example, in the manner of a tongue-and-groove connection, for which purpose they can have grooves and projections running in the direction of the longitudinal axis of the housing, which allow the partial housing parts to be brought together in the vertical direction along the longitudinal axis of the housing. In the cooperating position, the groove and projection can form a rear grip acting in a direction transverse to the longitudinal axis of the housing.

The outer partial housing part and the inner partial housing part resulting from the nesting can each partially form an area of the outer surface of the fourth housing part as a whole. It can be provided that the partial outer surfaces of the partial housing parts merge into each other at least approximately without steps.

The inner part-housing part may have a window part, for example for viewing a counter and/or a label of the received container. The window part may, as is also preferred, be exposed in an aperture area of the outer partial housing part.

Furthermore, such a two-part design can also be given with regard to the third, upper housing part.

In particular, but not exclusively, in the case of a nebulizer for multiple use, a locking pawl can be guided on the first housing part in a manner fixed to the direction of rotation, which pawl prevents the housing parts, in particular the first and second housing parts, from rotating relative to one another when the container is empty. In a selected position of a sleeve part surrounding the second housing part, which selected position is preferably the emptying position of the container, the locking pawl can enter a locking opening of the sleeve part by displacement in the direction of the longitudinal axis of the housing. Further rotation of the sleeve part and thus the relative rotation of the housing parts to each other is (at least temporarily) prevented. The locking pawl can be preloaded in the direction of the locking position by means of a spring, whereby the relevant spring is preferably supported on the third housing part.

The ranges or value ranges or multiple ranges indicated above and below also include all intermediate values with regard to the disclosure, in particular in 1/10 increments of the respective dimension, thus also dimensionless if applicable. For example, the specification 0.1 to 0.5-fold also includes the disclosure of 0.2 to 0.5-fold, 0.1 to 0.4-fold, 0.2 to 0.4-fold etc. On the one hand, this disclosure can serve to delimit a stated range limit from below and/or above, but alternatively or additionally also to disclose one or more singular values from a respective stated range.

### BRIEF DESCRIPTION OF THE DRAWINGS

The invention is explained below with reference to the enclosed drawing, which, however, only represents examples of embodiments. A part that is only explained with reference to one of the embodiments and is not replaced by another part in a further embodiment because of the special feature highlighted there is thus also described for this further embodiment as a part that is in any case possible. The drawing shows:

FIG. 1 the nebulizer, designed for single use, in perspective view, concerning a first embodiment with the cover cap closed;

FIG. 2 a representation view corresponding to FIG. 1, with the cover cap in the open position;

FIG. 3 the functional elements of the nebulizer for single use in an explosion perspective, such as a first inner housing part with a mouthpiece, a nozzle body, a retaining part, a counter and a second inner housing part;

FIG. 4 in an explosion perspective view, handling elements of the nebulizer of the first embodiment, such as a third outer housing part with the cover cap, a trigger button and a fourth outer housing part;

FIG. 5 a section through the cap along the line V-V in FIG. 1;

FIG. 6 a detailed explosion perspective view of a hinge area between the third housing part and the cover cap;

FIG. 7 the section along the line VII-VII in FIG. 5;

FIG. 8 the section along the plane VIII in FIG. 1, concerning a non-use position of the nebulizer;

FIG. 9 the magnification of the area IX in FIG. 8;

FIG. 10 the section along the line X-X in FIG. 8;

FIG. 11 a sectional view corresponding to FIG. 7, concerning a preparation position of the nebulizer;

FIG. 12 a continuation view of FIG. 11, concerning an atomisation position;

FIG. 13 the outer housing parts in a perspective view;

FIG. 14 the outer housing parts in a further perspective view;

FIG. 15 the outer housing parts in a sectional view according to FIG. 8, with inner casing parts indicated in dashed lines;

FIG. 16 a trigger ring in perspective;

FIG. 17 a perspective sectional view of the trigger ring, cut along the line XVII-XVII in FIG. 16;

FIG. 18 the section along the line XVIII-XVIII in FIG. 15;

FIG. 19 a perspective sectional view of the fourth housing part, cut along the line XIX-XIX in FIG. 18;

FIG. 20 the section along the line XX-XX in FIG. 12;

FIG. 21 a second embodiment of a single-use nebulizer in perspective view, with the cover cap closed;

FIG. 22 a representation corresponding to FIG. 21, with the cover cap in the open position;

FIG. 23 an exploded view of the handling elements of the nebulizer of the second embodiment;

FIG. 24 the section along the sectional plane XXIV in FIG. 21;

FIG. 25 a third embodiment of a single-use nebulizer in perspective view, with the cover cap closed;

FIG. 26 another perspective view of the nebulizer according to FIG. 25;

FIG. 27 a perspective sectional view, with the cover cap in the open position;

FIG. 28 an exploded view of handling elements of the nebulizer of the third embodiment;

FIG. 29 a cut along the sectional plane XXIX in FIG. 23;

FIG. 30 a fourth embodiment of a nebulizer designed for multiple use, shown in perspective, with the cover cap closed;

FIG. 31 another perspective view of the nebulizer shown in FIG. 30, with the cover cap in the open position;

FIG. 32 an exploded view of the functional elements of the nebulizer for multiple use;

FIG. 33 an exploded view of the handling elements of the nebulizer of the fourth embodiment;

FIG. 34 a section along sectional plane XXXIV in FIG. 29;

FIG. 35 a fifth embodiment of a nebulizer for multiple use in perspective view, with the cover cap closed;

FIG. 36 a representation corresponding to FIG. 31, but concerning the nebulizer according to FIG. 35;

FIG. 37 the nebulizer of the fifth embodiment in a perspective rear view;

FIG. 38 an exploded view of handling elements of the nebulizer of the fifth embodiment;

FIG. 39 a section along the section plane XXXIX in FIG. 34;

FIG. 40 a sixth embodiment of a nebulizer for multiple use in perspective view, with the cover cap closed;

FIG. 41 another perspective view of the nebulizer shown in FIG. 40;

FIG. 42 a perspective sectional view, with the cover cap in the open position;

FIG. 43 an exploded view of handling elements of the nebulizer of the sixth embodiment;

FIG. 44 a section along sectional plane XLIV in FIG. 40.

### DESCRIPTION OF THE EMBODIMENTS

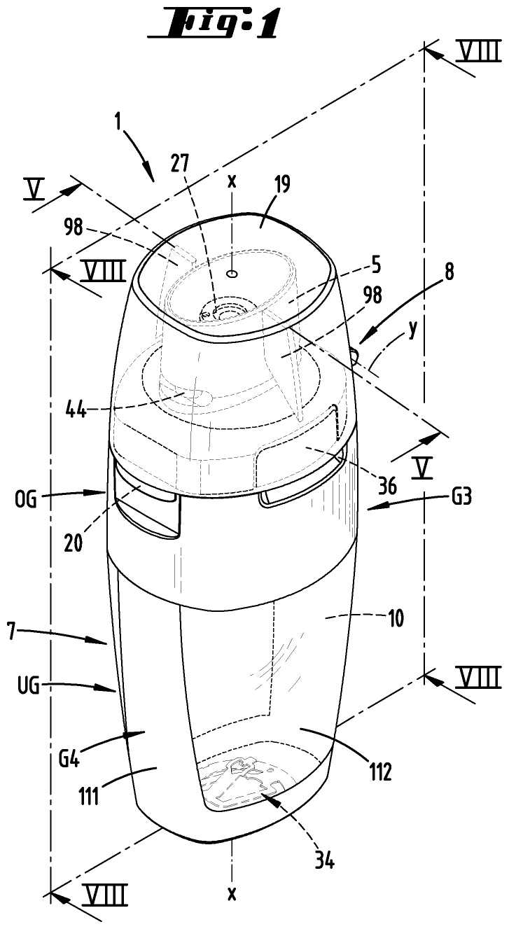

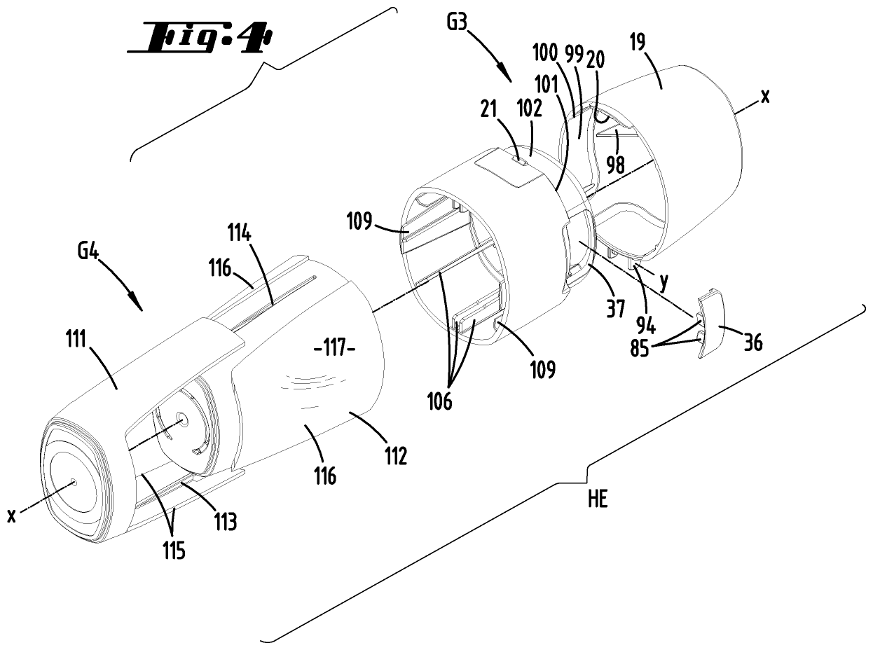

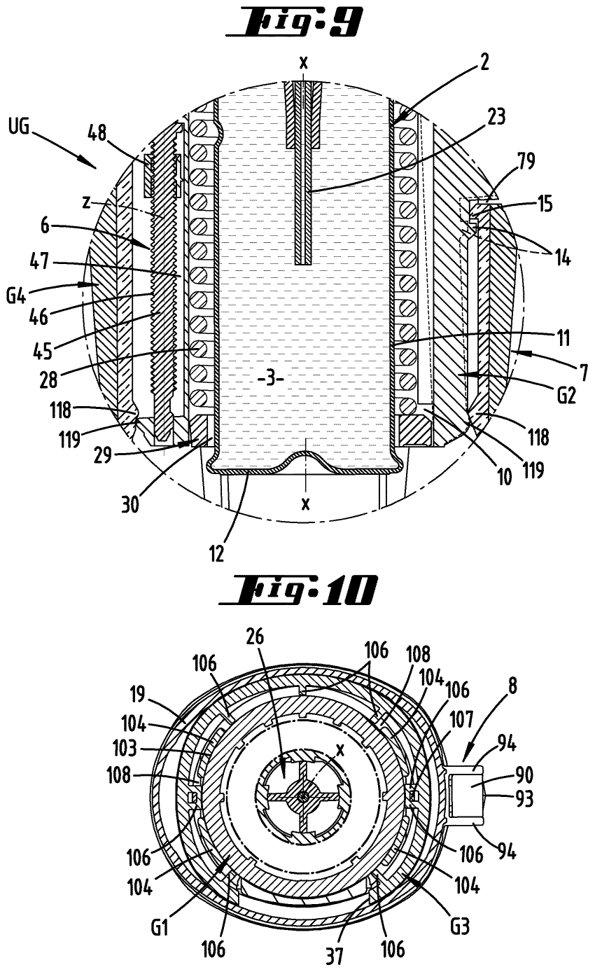

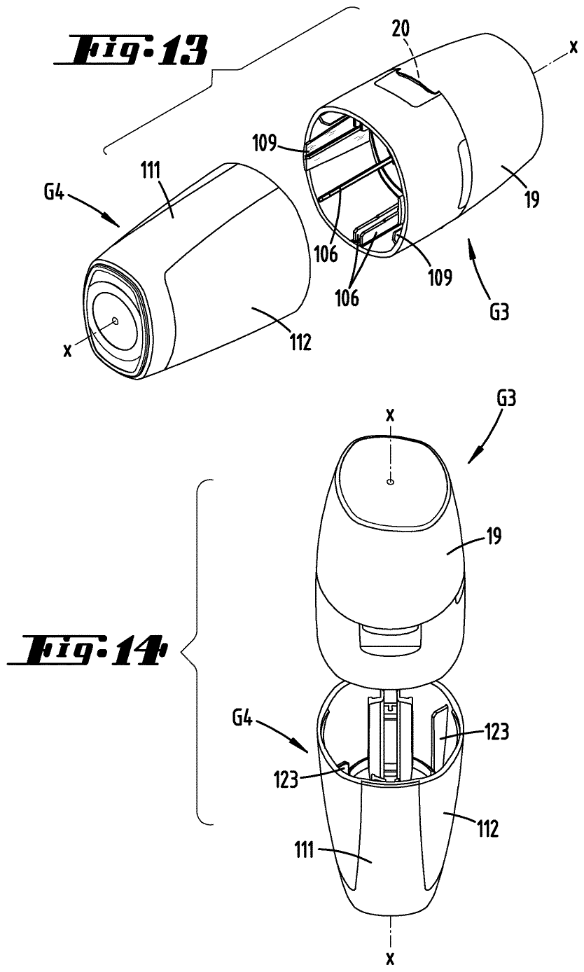

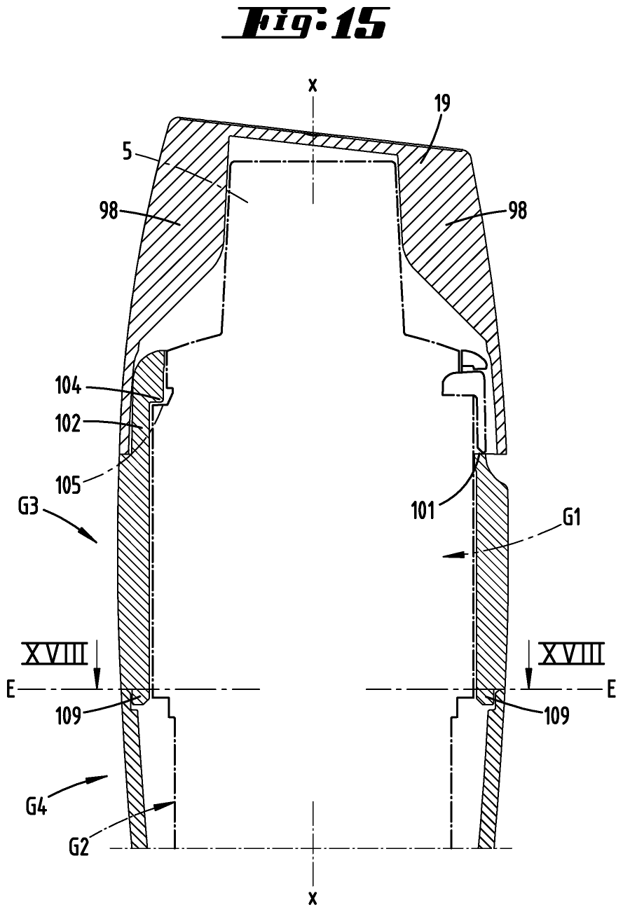

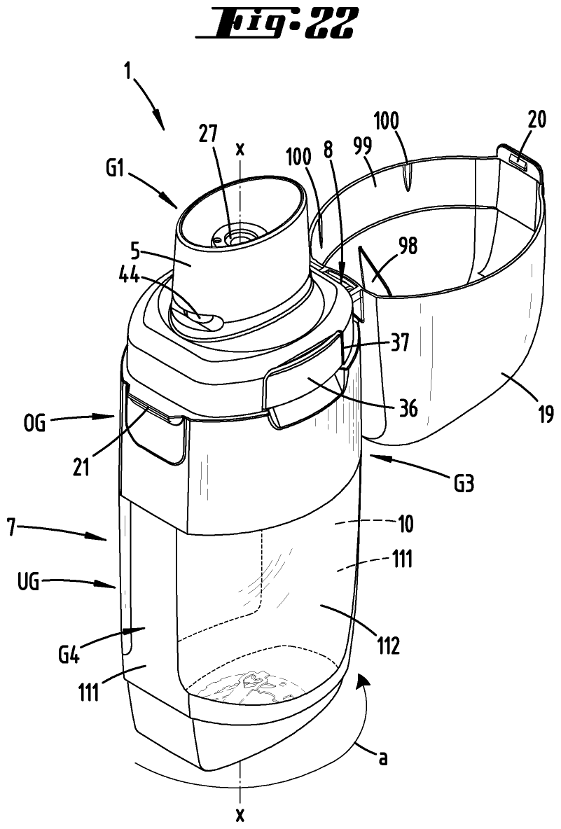

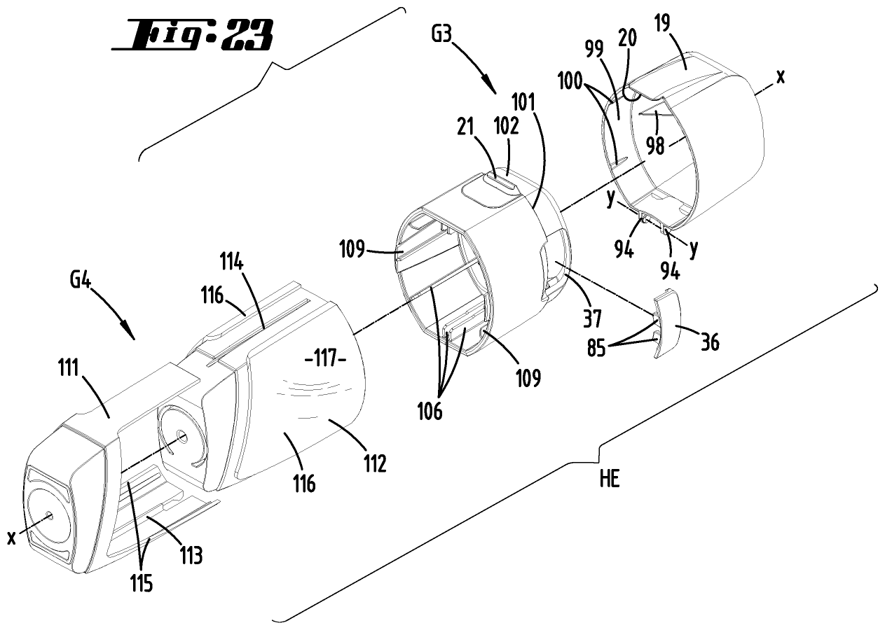

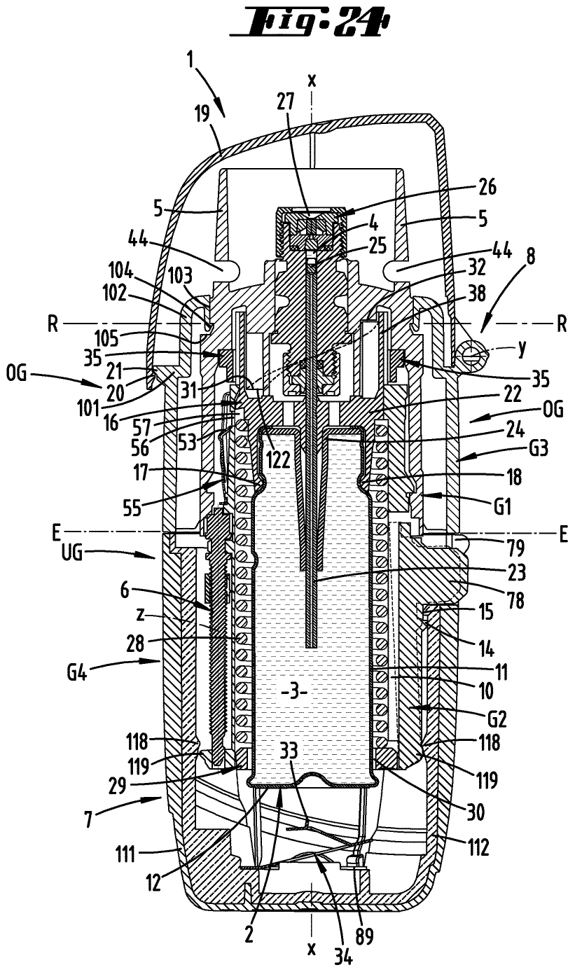

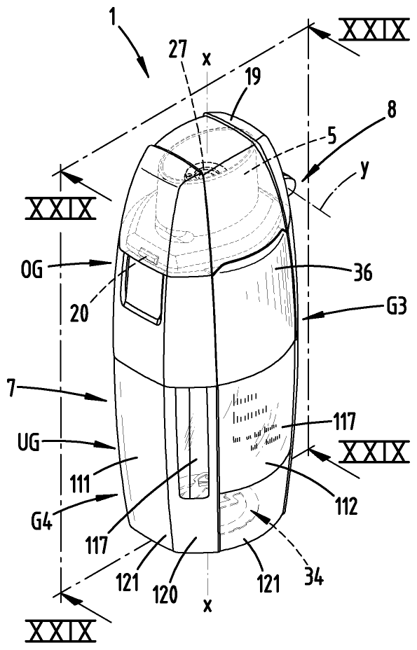

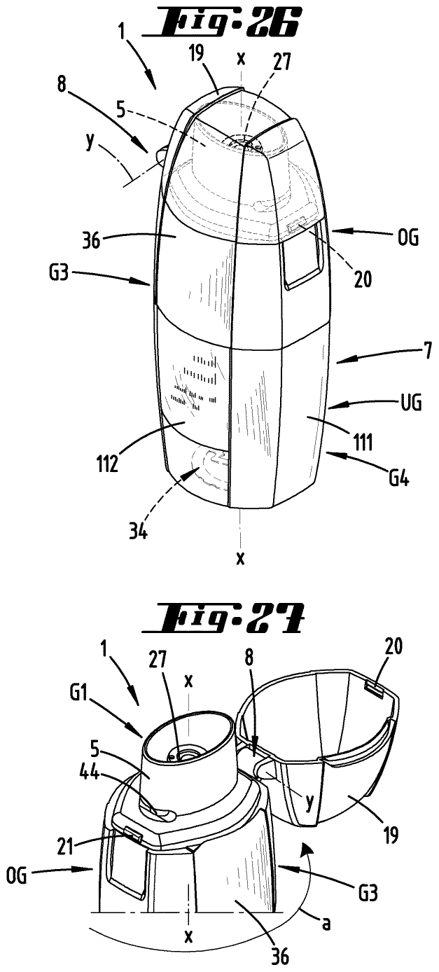

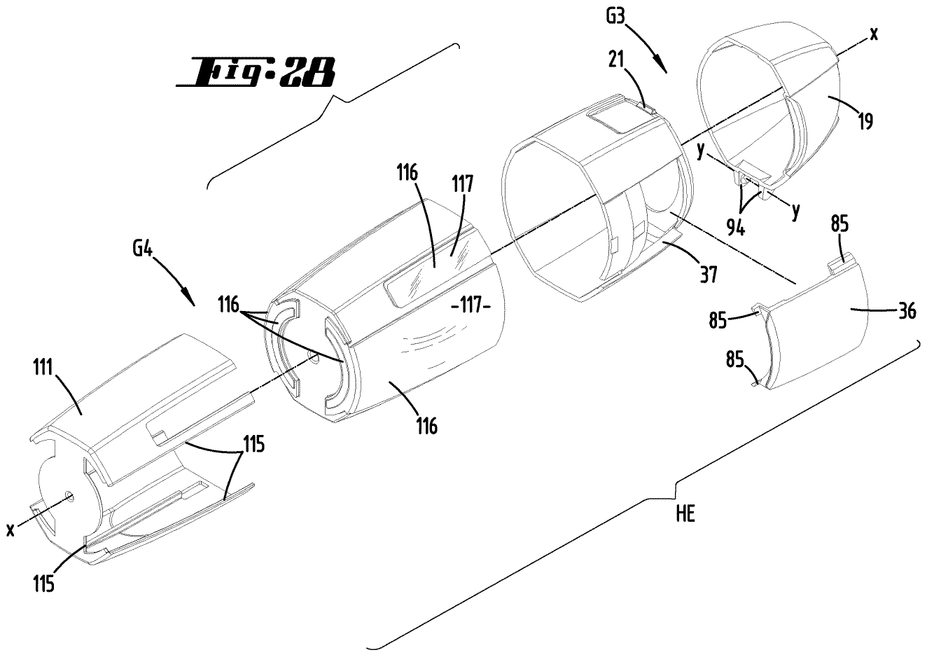

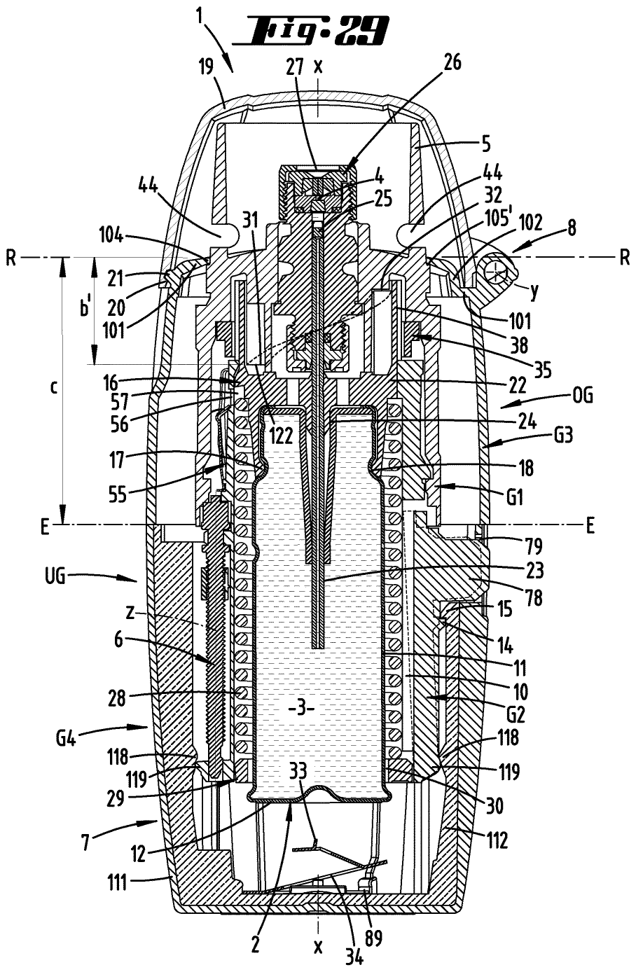

FIGS. 1 to 29 show embodiments of the nebulizer for preferred single use. FIGS. 1 to 20 relate to a first embodiment, FIGS. 21 to 24 to a second embodiment and FIGS. 25 to 29 to a third embodiment.

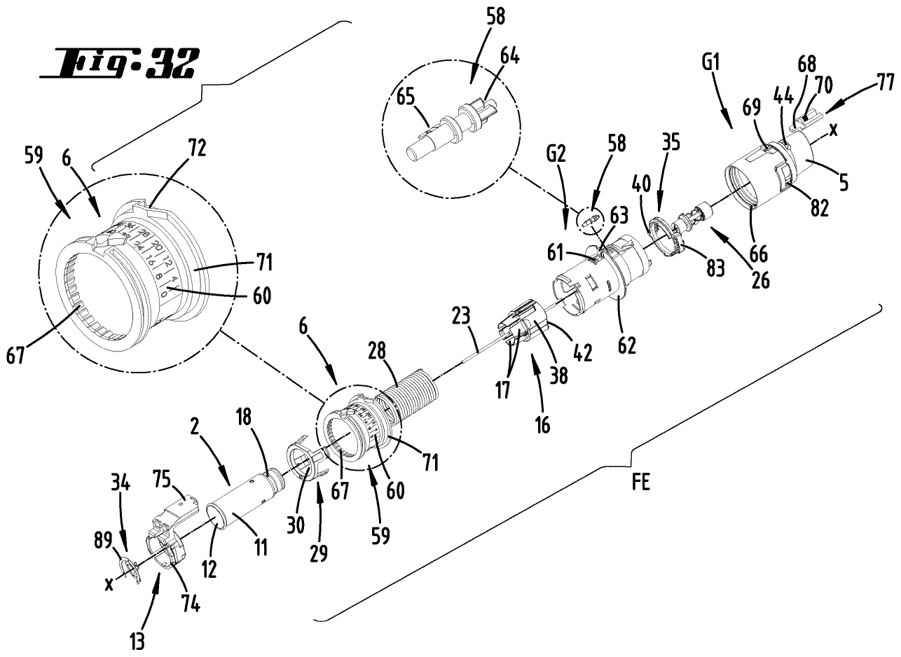

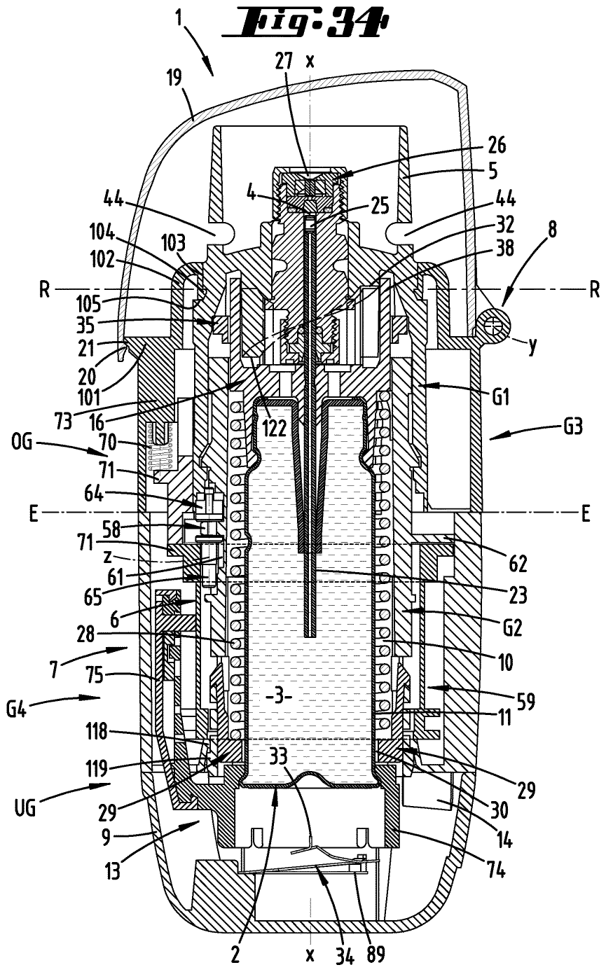

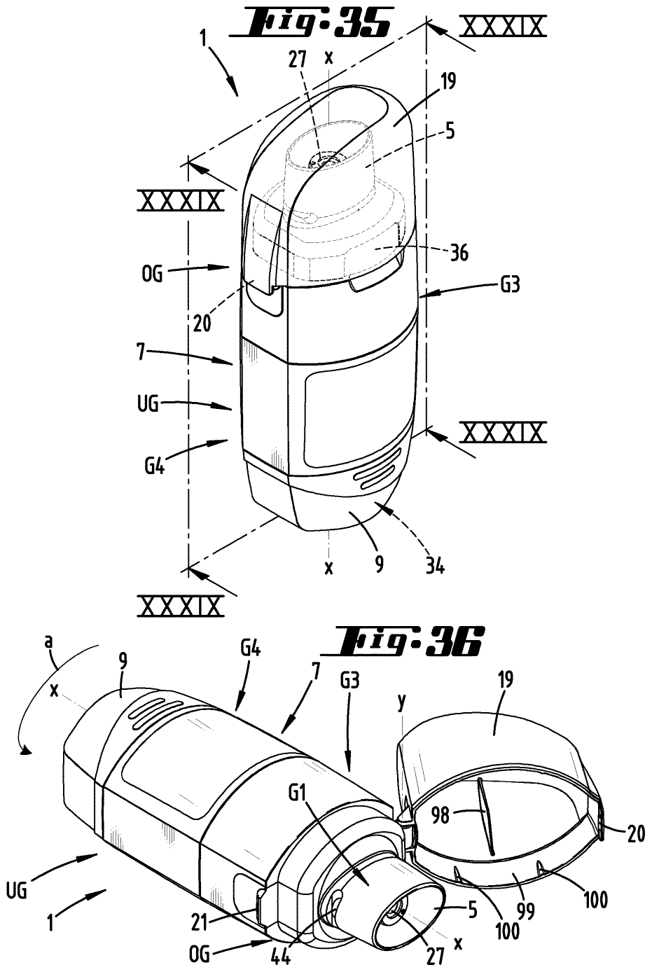

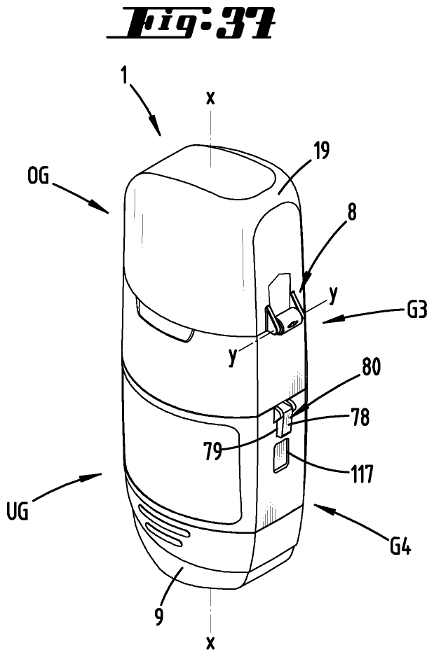

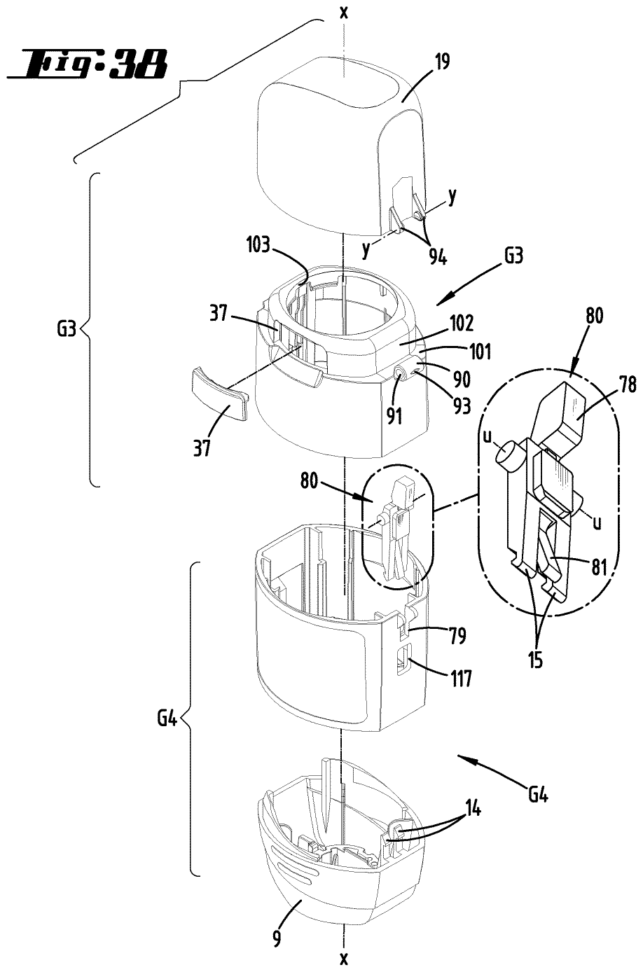

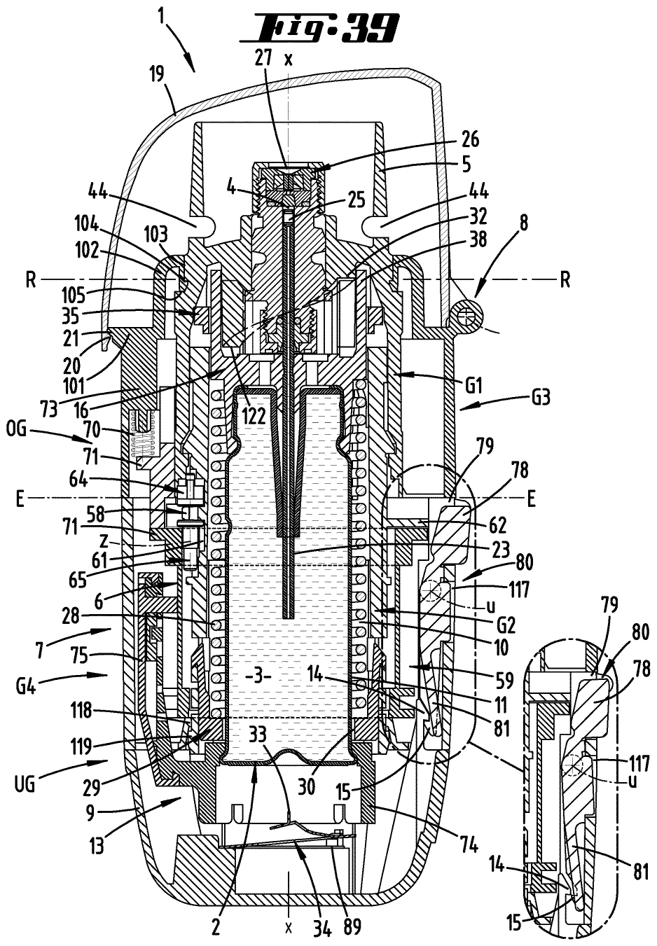

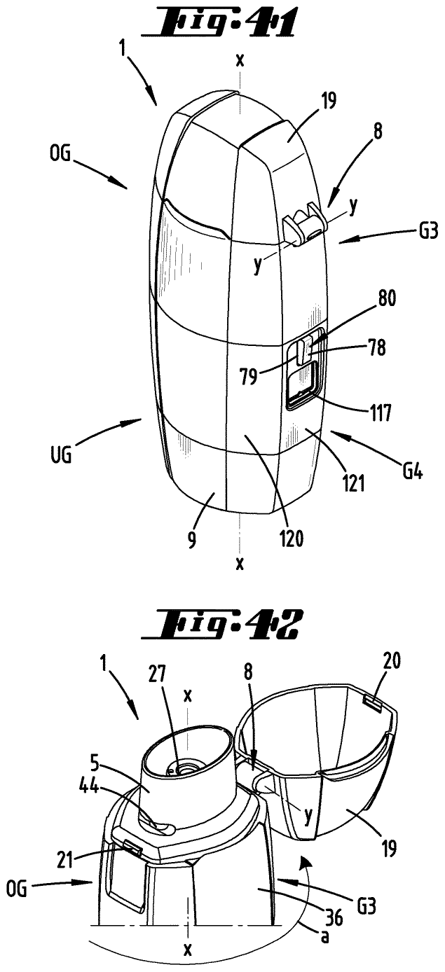

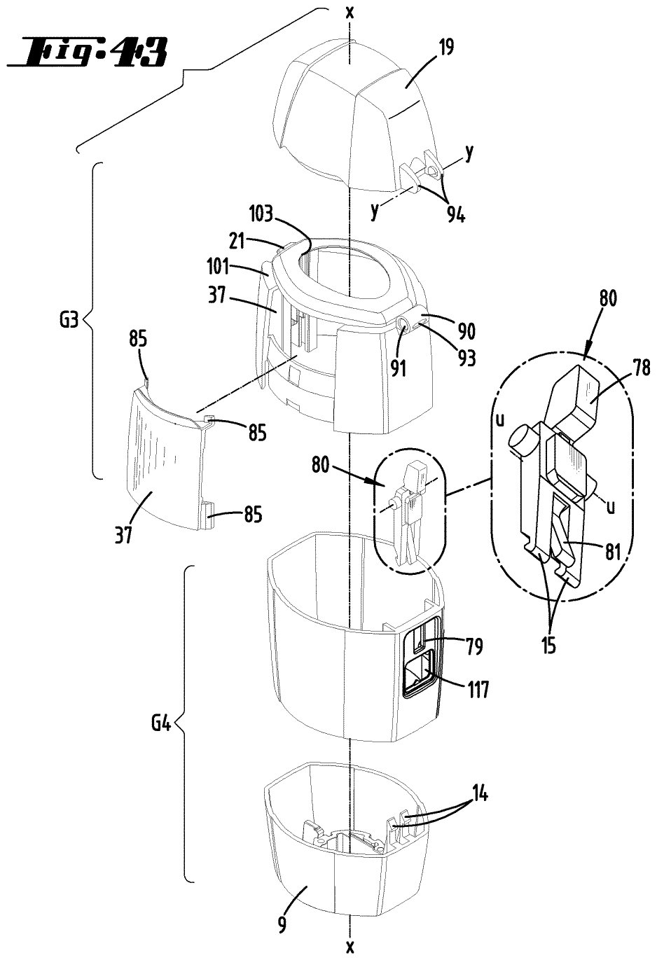

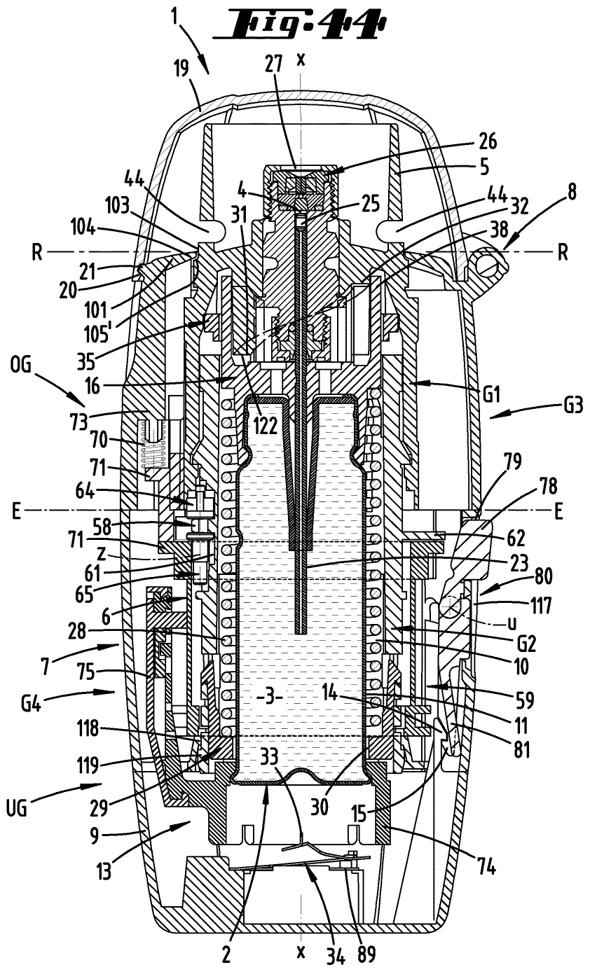

An embodiment of the nebulizer 1 with replaceable container 2 is shown in FIGS. 30 to 44, with a fourth embodiment example shown in FIGS. 30 to 34. FIGS. 35 to 39 concern a further fifth and FIGS. 40 to 44 a sixth embodiment.

The nebuliser 1 is preferably designed as a portable inhaler, further preferably having an elongated, round or oval cross-sectional shape, with a length of 80 to 200 mm, considered in the direction of a longitudinal housing axis x, further preferably about 100 to 150 mm, and a width or diameter of 20 to 60 mm, considered transversely thereto, further preferably about 25 to 50 mm. Accordingly, the nebulizer 1 can be guided and used with one hand, in particular when carrying out an inhalation process.

The fluid 3 is preferably a therapeutic or pharmaceutical product to be inhaled by the user during an inhalation process. Preferably, such an inhalation is carried out without propellant gas. Rather, the fluid 3 is preferably sucked from the container 2 into a pressure chamber 4 and, as a result of pressurisation, expelled in an atomising manner via a mouthpiece 5. The atomisation processes carried out can, as is also preferred, be counted using a counter 6 arranged on the housing side.

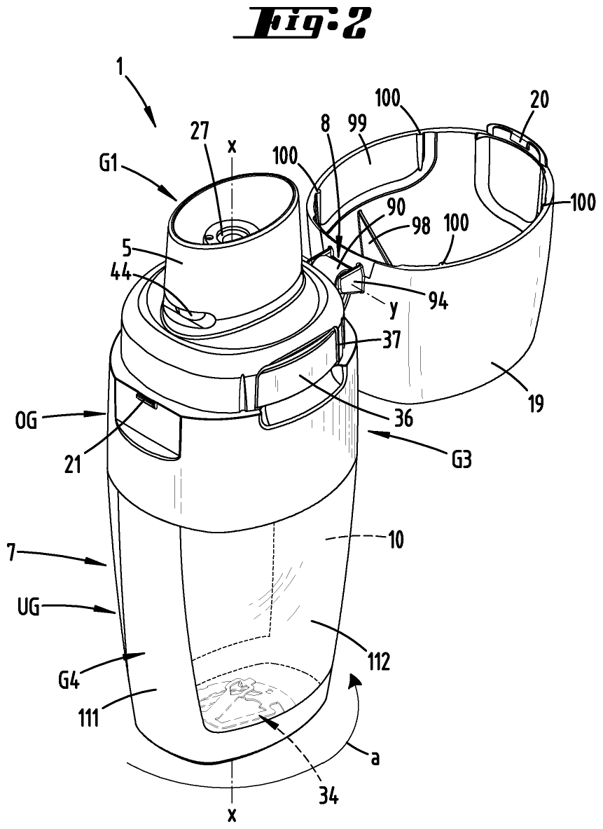

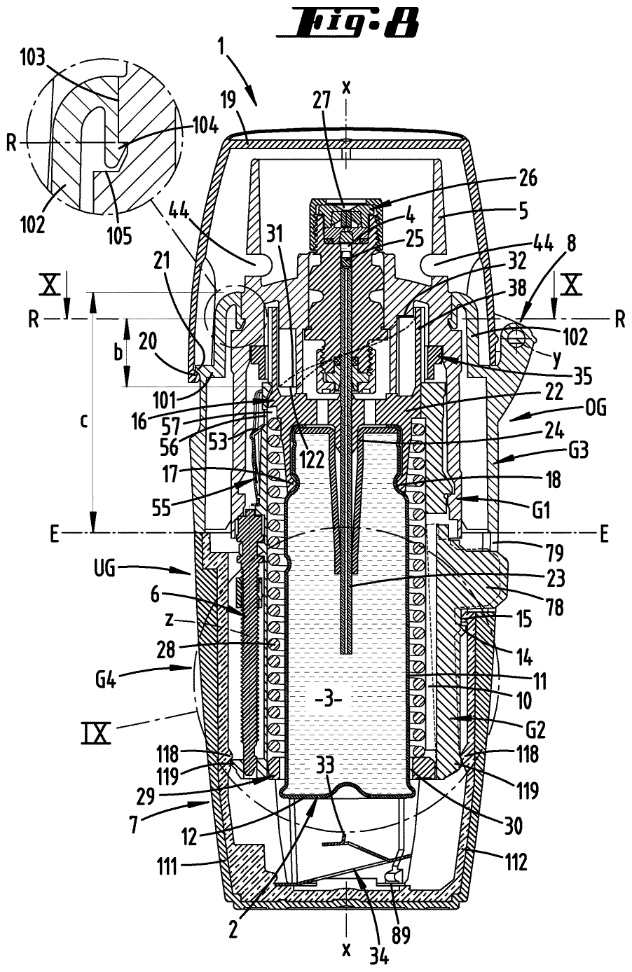

The nebulizer 1 has a housing 7 which is substantially subdivided into an upper housing part pair OG, comprising a first (upper) housing part G1 and a third (upper) housing part G3 which surrounds the first housing part G1 substantially in a pot-like manner, and a lower housing part pair UG, consisting of a second (lower) housing part G2 and a fourth (lower) housing part G4 surrounding the second housing part G2 substantially in a pot-like manner (compare, for example, FIGS. 8, 24, 29, 34, 39 or 44), wherein the housing part pairs OG and UG are rotatable relative to one another about the longitudinal housing axis x to a limited extent. For the description of the drawings, the first housing part G1 with the mouthpiece 5, which is preferably formed in one piece thereon, and the third housing part G3 are defined as fixed housing parts (correspondingly also the upper housing part pair OG), to which the second housing part G2 together with the fourth housing part G4 (and thus the lower housing part pair UG as a whole) can be rotated relative to one another about the longitudinal housing axis x (see direction of rotation a in FIG. 2, for example).

This results in a parting plane E running transversely to the longitudinal axis x of the enclosure, in which, visible from the outside, the outer housing parts G3 and G4—and thus coupled also the inner housing parts G1 and G2—facing each other in this parting plane E can be rotated relative to each other.

The container 2 is accommodated or can be accommodated in the housing 7 of the nebulizer 1, in particular in an receiving room 10 in the second housing part G2. The container 2 has a substantially circular-cylindrical or cartridge-like shape, whereby a collapsible, collapsible bag, in which the fluid 3 is held, is preferably accommodated in an outer rigid casing, substantially comprising a circumferential container wall 11 and a container bottom 12.

In a non-use position, as shown for example in FIG. 1, the mouthpiece 5 is preferably covered by a cover cap 19. The cover cap 19 is preferably held on the housing 7, in this case in particular on the third housing part G3, in a pivotally displaceable manner via a hinge joint 8. The geometric pivot axis y enabling this pivotal displacement can extend essentially perpendicular to the longitudinal axis x of the housing. In the cover cap closed position shown in FIG. 1, for example, the cover cap 19 can engage behind a counter latching section 21 provided on the housing side by means of a latching section 20 to secure the closed position.

The cover cap 19 may be part of the third housing part G3, as may a trigger button 36 described in more detail below.

To form the hinge joint 8, a hinge bracket 90 can be integrally formed on the outside of the wall of the third (upper) housing part G3, having a hinge eye 91. The walls delimiting the hinge bracket 90 in the direction of the pivot axis y can each have a recess 92 in a region between the hinge eye 91 and the facing outer surface of the third housing part G3, for example to make the housing part G3 more economical to manufacture by reducing the wall thickness. Away from the otherwise circumferential housing wall of the third housing part G3, an over-flowable latching bead 93 can be formed on the outside of the hinge bracket 90.

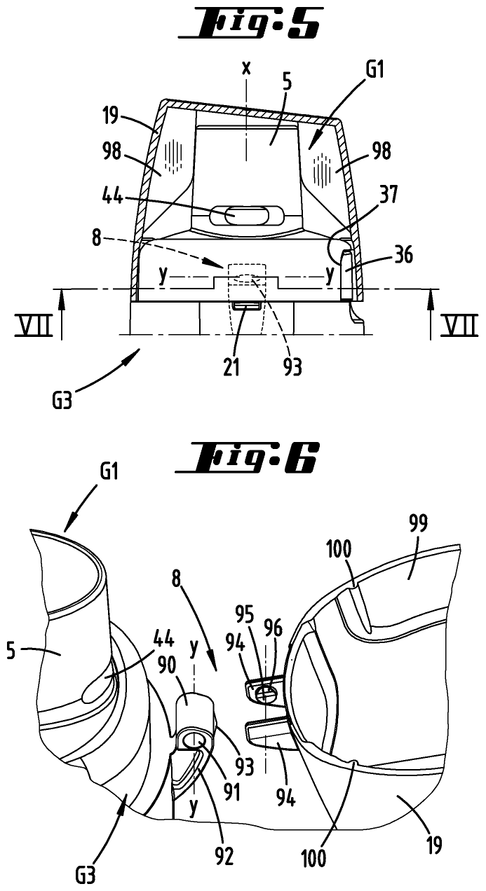

Furthermore, two hinge arms 94 spaced apart from each other in the direction of the pivot axis y are integrally formed on the cover cap 19 to form the hinge joint 8. The axial spacing is adapted to the axial dimension of the hinge bracket 90. A hinge pin 95 is formed on each of the inner surfaces of the hinge arms 94 facing one another, which hinge pin 95 preferably has a circular outline substantially adapted to the hinge eye 91. The hinge pin 95 can be provided with an overflow slope 96 to simplify assembly (compare FIG. 6).

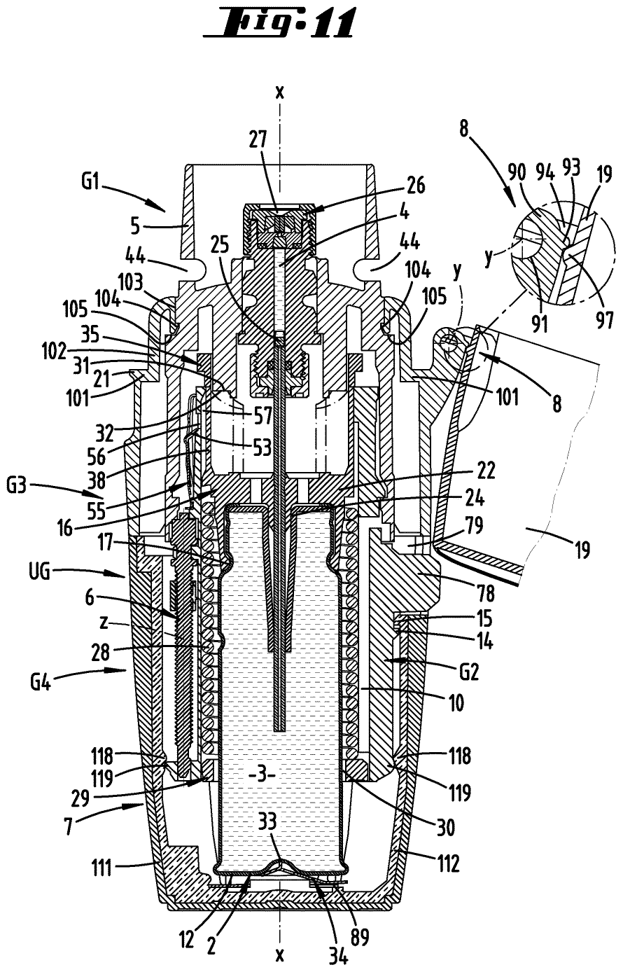

As can be seen from the magnifying glass in FIG. 11, a cover cap open position can be snap-locked in that a counter bead 97 of the cover cap 19 near the hinge overlaps and engages behind the latching bead 93 on the hinge bracket side.

Alternatively, a substantially one-piece hinge bracket with a hinge eye can also be provided on the cap side, while hinge arms with hinge pins that are spaced apart from each other in the direction of the pivot axis y can be moulded onto the outer wall of the third housing part G3.

Also, on the inside of the wall of the cover cap 19, inwardly projecting support ribs 98 may be formed which, in the cap-closing position, may bear against the outside of the wall of the mouthpiece 5 and thus contribute to centring the cover cap 19 relative to the mouthpiece 5 and to stabilising the cover cap 19 as such.

In the area of an end region facing the mouthpiece 5, the container 2 is snap-fitted to a retaining part 16 which is guided in the second housing part G2 linearly along the longitudinal axis x of the housing and non-rotatably relative to the second housing part G2. Spring-loaded, radially inwardly directed latching projections 17 of the retaining part 16 engage in a circumferential latching groove 18 of the container 2 in the area of its container neck.

In the embodiments relating to the disposable nebulizer 1 (FIGS. 1 to 29), the pot-shaped fourth housing part G4 is non-rotatably connected to the second housing part G2 in the usual position of use, as shown for example in FIGS. 1 and 2. After the fourth housing part G4 has been removed, for example by releasing a latch between a latching projection 14 formed on the second housing part G2 and a facing latching shoulder 15 of the housing part G4, which release can preferably be carried out without tools by the user if required, the container 2 can be inserted from below into the housing 7, in particular into the second housing part G2 or into the receiving room 10, prior to initial use.

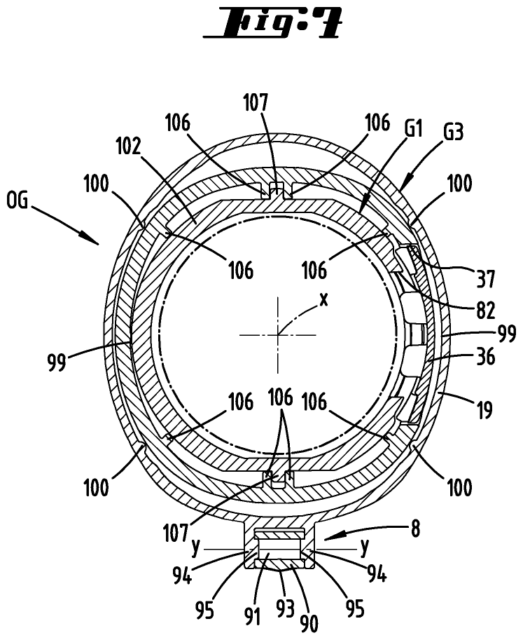

To release the latch between the second housing part G2 and the fourth housing part G4, the user actuates a key projection 78 which is formed on the second (inner) housing part G2, passes through an outwardly open slot opening 79 of the fourth (outer) housing part G4 and projects freely outwards, which, with a resilient inward deflection (see dotted line illustration in FIGS. 7, 24 and 29), clears the way for the container connection part 13 to be pulled off along the axis x—with reference to the usual downward orientation of the nebulizer 1.

In addition to a latching of the housing parts G2 and G4 between the latching projection 14 and the latching shoulder 15, as is also preferred, at least one further latch can be provided which can preferably be overrun by the action of a force in the axial direction. For this purpose, latching projections 118 can be formed on the inside of the wall of the outer housing part G4 for latching cooperation with counter latching projections 119 on the inner housing part G2.

In the embodiments of a nebulizer 1 for use with exchangeable containers 2 shown in FIGS. 30 to 44, the fourth housing part G4, which is sleeve-shaped in this case, basically remains on the second housing part G2. For this purpose, latching projections 118 are also formed on the inside of the wall of the outer housing part G4, but here for latching cooperation with counter latching projections 119 of the inner housing part G2, which preferably cannot be overrun.

In order to load a container 2 or to remove an (emptied) container 2, if necessary together with a container connection part 13, in the embodiment examples of FIGS. 30 to 44 a closure cap 9 on the foot side is pulled downwards essentially along the axis x. The closure cap 9 can be snap-fitted in the simplest way on the fourth housing part G4, as shown in FIG. 34. The closure cap 9 can, as shown by the fourth embodiment example in FIG. 34, be held in a latching manner on the fourth housing part G4 in a very simple manner, cap-side latching projections 14 can cooperate with latching shoulders formed on the fourth housing part G4 for this purpose. The retaining forces of the latch can be overcome by pulling the closure cap 9 downwards.

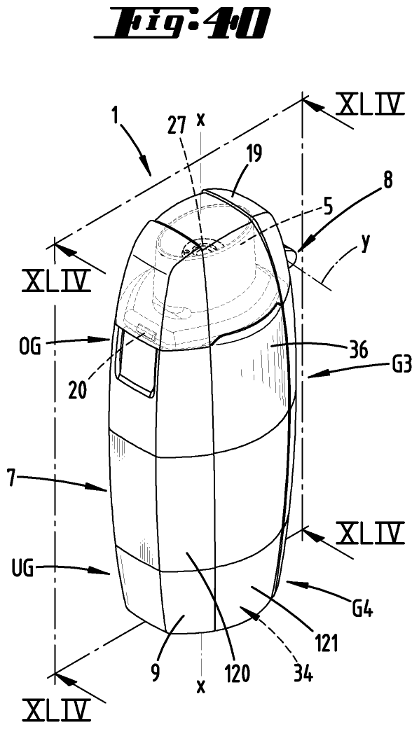

Alternatively, according to the fifth and sixth embodiments, a release lever 80 can also be provided for releasing the latch between the fourth housing part G4 and the closure cap 9, which is held on the fourth housing part G4 so as to be pivotable about an axis u preferably aligned in the same direction as the pivot axis y of the cover cap 19. A key projection 78 of the release lever 80 projects freely outwards for actuation, passing through a slot opening 79 of the fourth housing part G4. A latching shoulder 15 is formed on the release lever 80 at the end facing away from the key projection 78, with a spring arm 81 extending adjacent to it. The spring arm 81 is supported on the inside of the wall on the fourth housing part G4 and loads the latching shoulder 15 in the direction of the latching position according to FIGS. 39 and 44, in which latching position the latching shoulder 15 catches behind a latching projection 14 of the closure cap 9. The latching of the closure cap 9 is released by pressing the release lever 80 and the associated pivoting displacement of the release lever 80 with the latching shoulder 15.

The retaining part 16 serves in particular for dispensing and atomising the fluid 3, preferably in a defined dosing quantity. For this purpose, the retaining part 16 has, in addition to the holder 22 for the container 2, which has the latching projections 17, a suction tube 23 which, when the container 2 is latched to the holder 22, dips into the interior of the container, in particular into the interior of the bag held in the container 2, while piercing a passage opening 24 of the container 2.

The end of the suction pipe 23 facing away from the container 2 and pointing upwards in the illustrations can be provided at the end with a non-return valve 25, whereby this end region of the suction pipe 23 lies in the pressure chamber 4 formed in a nozzle body 26 so as to be linearly displaceable along the longitudinal axis x of the housing. Viewed in the direction of extension of the longitudinal axis x of the housing, an ejection nozzle 27 can be formed at the end of the pressure chamber 4 associated with the mouthpiece 5.

The retaining part 16 can be spring-loaded in the direction of the mouthpiece 5, preferably with a limited stop. For this purpose, as is also preferred, a compression spring 28, for example in the form of a cylinder compression spring, can be provided, which preferably extends concentrically to the longitudinal axis x of the housing. The compression spring 28 is supported in the region of one end on the underside of the retaining part 16, surrounding the holder 22, and in the region of the opposite and, according to the illustrations, downwardly pointing end on a base part 29 latched to the second housing part G2. This base part 29 is provided with a central opening 30 through which the container 2 can project downwardly into the region of the container connection part 13 or through which opening 30 the container 2 can be inserted for latching to the holder 22.

In preparation for an atomisation or inhalation process, the compression spring 28 is tensioned, i.e. its axial extension is compressed. For this purpose, the lower housing part pair UG is rotated relative to the upper housing part pair OG about the longitudinal housing axis x, preferably by a rotation angle of about 180°. In the process, a helical bevel surface 31 formed on the underside of the first housing part G1, facing away from the mouthpiece 5, interacts with a facing counter bevel surface 32 which likewise slopes downwards or upwards in the circumferential direction. of the retaining part 16, so that in the course of the rotation of the lower housing part pair UG relative to the upper housing part pair OG, a linear displacement of the retaining part 16 with the container 2 fixed thereto is achieved along the longitudinal axis x of the housing with reference to the representation in FIG. 11 downwards in the direction of the base part 29 against the restoring force of the compression spring 28. In the course of this linear displacement, fluid 3 is simultaneously sucked or conveyed from the container 2 via the suction pipe 23 into the pressure chamber 4, which successively enlarges in the course of the displacement of the retaining part 16.

The helical bevel surface 31 preferably ends, with respect to the direction of extension of the longitudinal axis x of the housing, above and at a distance from the parting plane E.

To equalise the pressure in the container 2, the container bottom 12 is pierced, in particular in the course of the first use of the container 2, during the downward displacement described above, in order to tension the system, for which purpose a piercing needle 33, preferably spring-supported in the direction of the longitudinal axis x of the housing, is provided on the base side of the housing part G4 or the closure cap 9 (compare FIG. 11). As shown, the piercing needle 33 can be part of a spring plate member 34 held and formed in the fourth housing part G4 or in the closure cap 9, so that the piercing needle 33 is spring-biased in the direction of the container bottom 12.

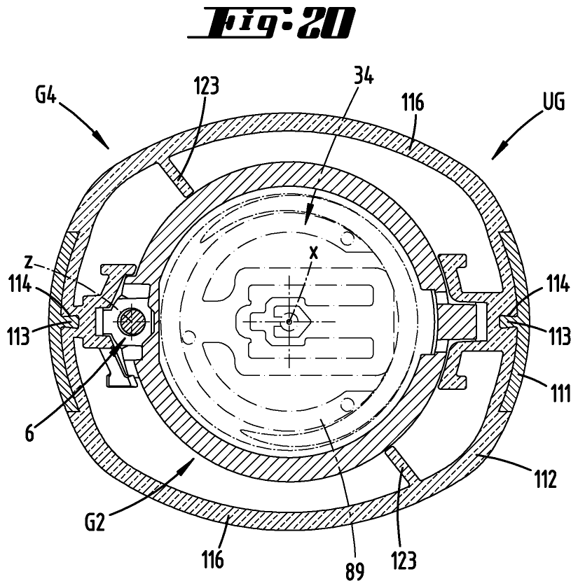

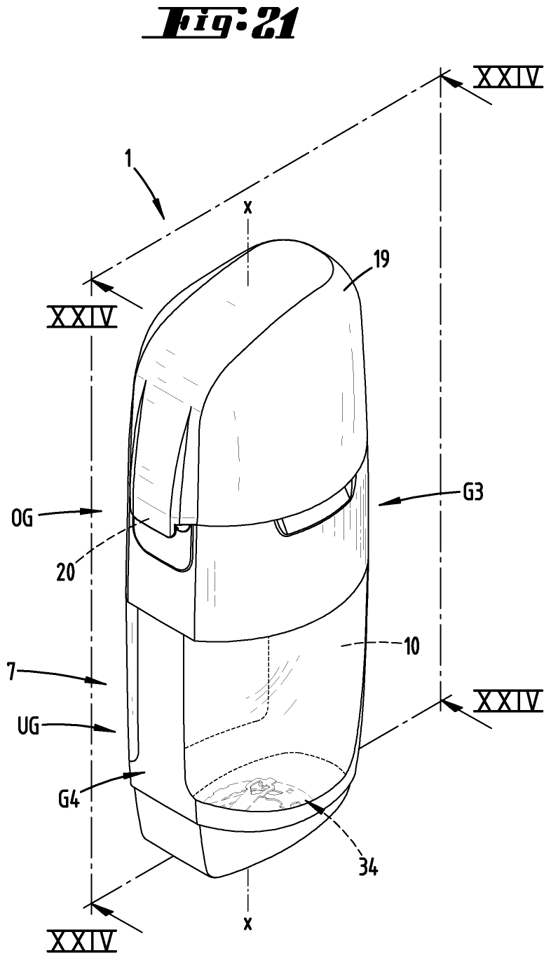

The spring plate member 34 can be snap-fitted on the bottom side of the closure cap 9 or of the fourth housing part G4. For this purpose, latching ribs 88 can be formed on the closure cap 9 or on the fourth housing part G4, which in the latching position engage over the edge of an annular base section 89 of the spring plate member 34 (compare FIGS. 18 and 19).

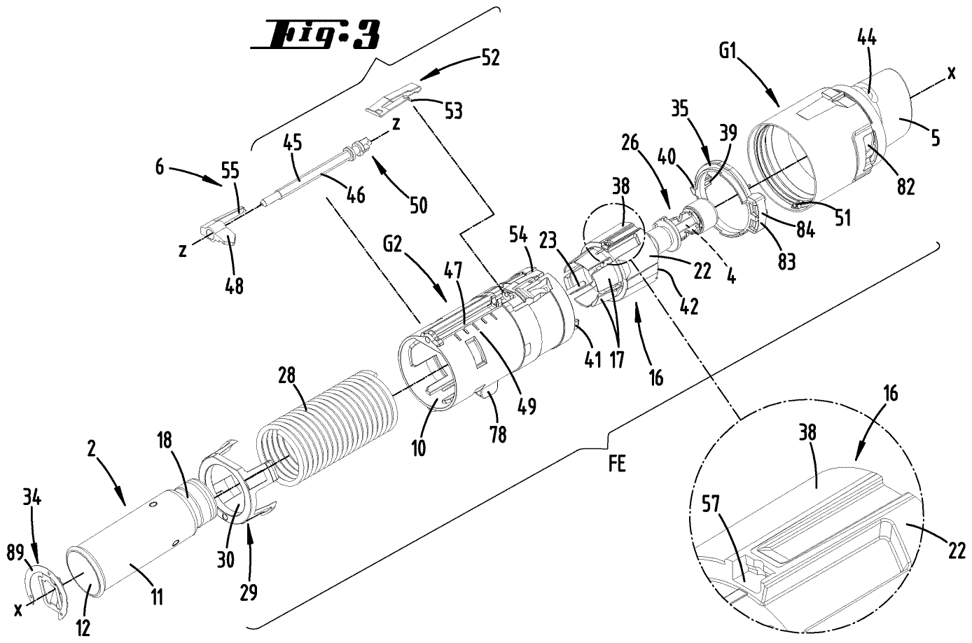

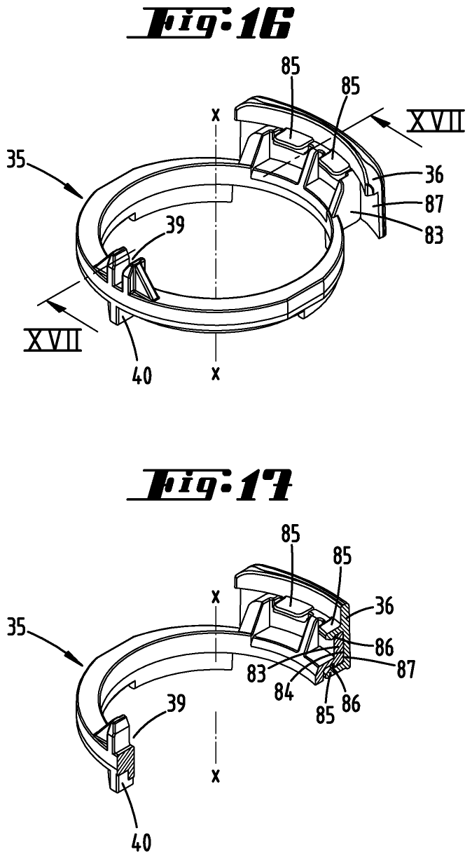

The tensioned position of the retaining part 16 is initially secured. In particular, a trigger ring 35 arranged in the first housing part G1 serves this purpose, which can be acted upon from the outside by the user via a trigger button 36. For this purpose, the trigger button 36 is exposed in a window 37 of the third housing part G3, passing through an opening 82 in the first housing part G1 (compare FIGS. 3 and 16).

In a non-use position of the nebulizer 1, the trigger button 36 may be at least partially, but preferably completely, covered by the cover cap 19 and held in a protected hidden position.

Rib-like projections 100 can be formed on the inside of the wall, approximately at the end of this cap section 99 as viewed in the circumferential direction—preferably on both sides—associated with the cap section 99 of the cover cap 19 which covers the trigger button 36 in the cap closed position, for example as shown in FIG. 1, and also preferably essentially diametrically opposite this cap section 99. These serve in particular to centre the cover cap 19 relative to the third housing part G3 in the cap closed position. In addition, in the cap closed position, the resulting support of the projections 100 on the facing wall of the third housing part G3 can prevent actuation of the trigger button 36 by pressure on the cover cap 19, for example in the region of its cap section 99.

The trigger ring 35 and the trigger button 36 can be formed in two parts, as also shown, whereby a design adaptation of the trigger button 36, for example with regard to the colour design or the material used and/or with regard to the external dimensions, to the design in particular of the outer third (upper) housing part G3 and/or of the housing-side window 37 is made possible with a preferably basically constant trigger ring 35.

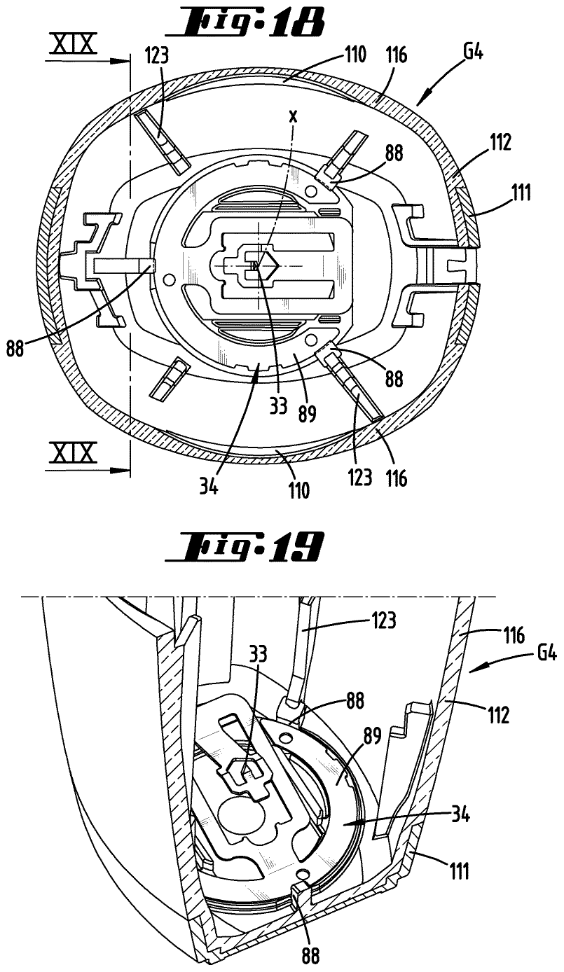

As can be seen, for example, from the individual illustrations in FIGS. 16 and 17, a retaining base 83 for the trigger button 36 can be moulded onto the trigger ring 35, associated with the opening 82 in the first housing part G1. This retaining base 83 provides an outer contact surface 84 for a facing inner support surface 87 of the separately provided trigger button 36.

Facing this support surface 87 on the button side, latching projections 85 are formed on the trigger button 36 for latching cooperation with the retaining base 83, whereby in the latching position the latching projections 85 both engage over and under the section with the contact surface 84. The latching projections 85 engage in exposed latching receptacles 86 of the retaining base 83 (compare FIG. 17).

Viewed in the circumferential direction, the trigger button 36 can extend beyond the retaining base 83 on both sides (compare FIG. 16). Viewed in the axial direction, the trigger button 36 may extend beyond the retaining base 83 in one direction only—preferably upwards—as shown.

The trigger ring 35 initially and substantially surrounds a wall 38 of the retaining part 16 surrounding the region of the counter bevel surface 32, with an increase in the thickness of the ring measured in the radial direction, viewed from both sides in the circumferential direction from the region of the trigger button 36. In the region essentially diametrically opposite the trigger button 36, the trigger ring 35 has a receptacle 39 (see also FIG. 3) for a housing-side rib which opens upwards in a U-shape with respect to an orientation of the nebulizer 1 as shown in FIG. 8, as a result of which the trigger ring 35 is held in the first housing part G1 in a manner secured against rotation.

With reference to the illustrations, the receptacle 39 is formed on the top side of the trigger ring 35. A control projection 40 is formed on the underside of the trigger ring 35 approximately overlapping in the direction of the longitudinal axis x of the housing. During preparation of the nebulizer 1 by relative rotation of the housing part pairs OG and UG in the circumferential direction, this projection interacts with a counter control projection 41 of the second housing part G2, which rotates in the course of the rotation relative to the fixed trigger ring 35, in such a way that a radially inward loading of the trigger ring 35 in the region of the control projection 40 is achieved via the sloping surfaces of the projections sliding against each other.

Due to the relative rotation of the housing part pairs OG and UG with respect to each other, a relative rotation of the retaining part 16 with respect to the fixed trigger ring 35 is also achieved in this position, so that the control projection 40 can enter radially inwards into a recess 42 formed on the front side of the wall 38 of the retaining part 16.

If necessary, with overrunning of the counter control projection 41, the control projection 40 can partially engage in the recess 42 in order to lock the tensioned position, thereby bearing against a shoulder of the retaining part 16 which delimits the recess 42 in the circumferential direction, as well as, if necessary, further or alternatively thereto, bearing with a radial shoulder of the projection spaced in the circumferential direction of the trigger ring 35 from the helical bevel surface of the control projection 40 against an associated radial shoulder of the counter control projection 41, which can be reached after overrunning.

This firstly provides a safeguard to prevent reverse rotation and/or further rotation of the lower housing part pair UG relative to the upper housing part pair OG in the tensioned position.

In the clamping direction, the annular control projection 40 is overlapped by the counter control projection 41.

In addition, the tension of the trigger ring 35 when the retaining part 16 is displaced downwards, possibly supported by the radial displacement of the control projection 40 by the counter control projection 41, which may be due to the design of the trigger ring 35 with a different thickness in the circumferential direction, can result in an inward displacement of the trigger ring 35—preferably together with the control projection 40, in particular in this region diametrically opposite the trigger button 36 and having the control projection 40, an inward displacement of the trigger ring 35—preferably together with the control projection 40—in a radially inward direction, so that the relevant section of the trigger ring 35 lies in front of the facing end face of the wall 38 of the retaining part 16 in a locking manner in the axial direction or engages in the recess 42. engages in the recess 42 and locks in front of the bottom of the recess 42.

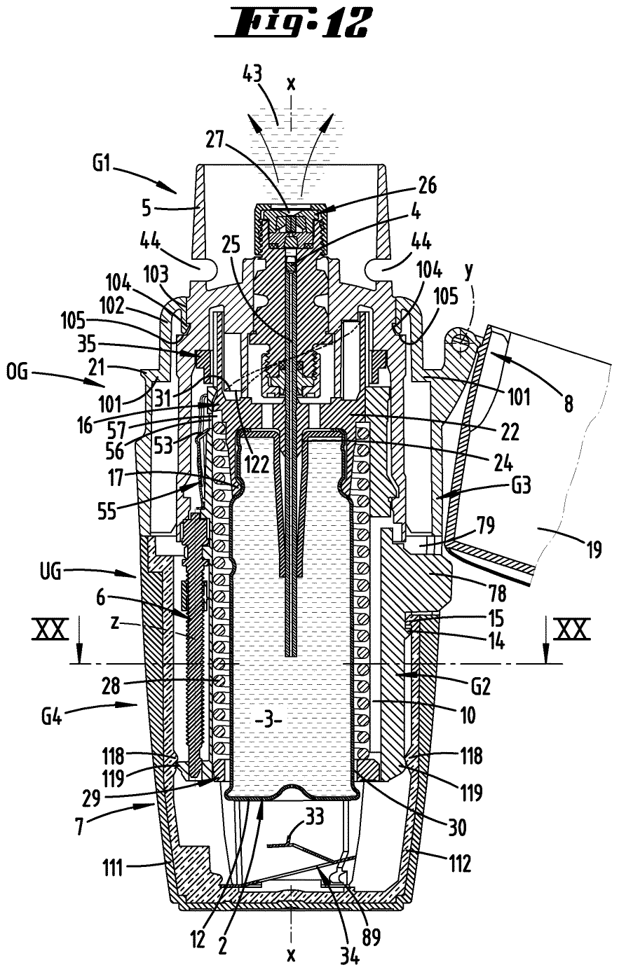

For inhalation, the user moves the cover cap 19 to a position exposing the mouthpiece 5, for example as shown in FIG. 2, and surrounds the mouthpiece 5 with the lips. With the subsequent release of the compression spring 28, the fluid dose divided in the pressure chamber 4 is expelled via the ejection nozzle 27 (compare FIG. 12). The user can inhale the emerging aerosol 43, for which purpose air can be sucked in via at least one ventilation opening 44 at the foot side of the mouthpiece 5.

Unlocking is effected by a pressure actuation of the trigger button 36 by the user, whereby the trigger ring 35 is loaded in such a way that its area essentially opposite the trigger button 36, in particular having the control projection 40, is displaced back radially outwards again, which leads to a release of the retaining part 16. Accordingly, this can spring back into the starting position as a result of the release of the restoring force of the compression spring 28, whereby the fluid is expelled from the pressure chamber 4 and via the ejection nozzle 27 via the now closed non-return valve 25, which serves quasi as a piston in the course of this process. This can result in a nozzle jet fan of the aerosol 43 as shown in FIG. 12, with an opening angle of about 30 to 150 degrees, further about 75 to 115 degrees.

An atomising operation can be registered and counted using a counter 6 housed in the housing 7. The counting takes place with the rotation of the housing part pairs OG and UG relative to each other, i.e. with the preparation of the nebulizer 1 for carrying out a fluid discharge. An action on the counter 6 in the sense of a count does not necessarily have to take place each time the housing part pairs OG and UG are rotated relative to one another. For example, only every second or every fourth rotation can lead to an action on the counter 6 in the sense of a count.

As a result of the design and arrangement of housing part pairs OG and UG, the nebulizer components are advantageously divided into “inner” functional elements FE and “outer” handling elements HE, the handling elements HE essentially comprising the upper third housing part G3 with the cover cap 19 pivotably mounted thereon, the trigger button 36 and the lower fourth housing part G4, optionally together with the closure cap 9 and further optionally with the spring plate member 34 having the piercing needle 33. The functional elements FE comprise the further “inner” components, such as essentially the first housing part G1 with the mouthpiece 5, the second housing part G2, the retaining part 16 with the suction pipe 23, the nozzle body 26, the trigger ring 35, the compression spring 28, the base part 29 and the counter 6, if provided. In a functional nebulizer 1, part of the functional elements FE is also the container 2.

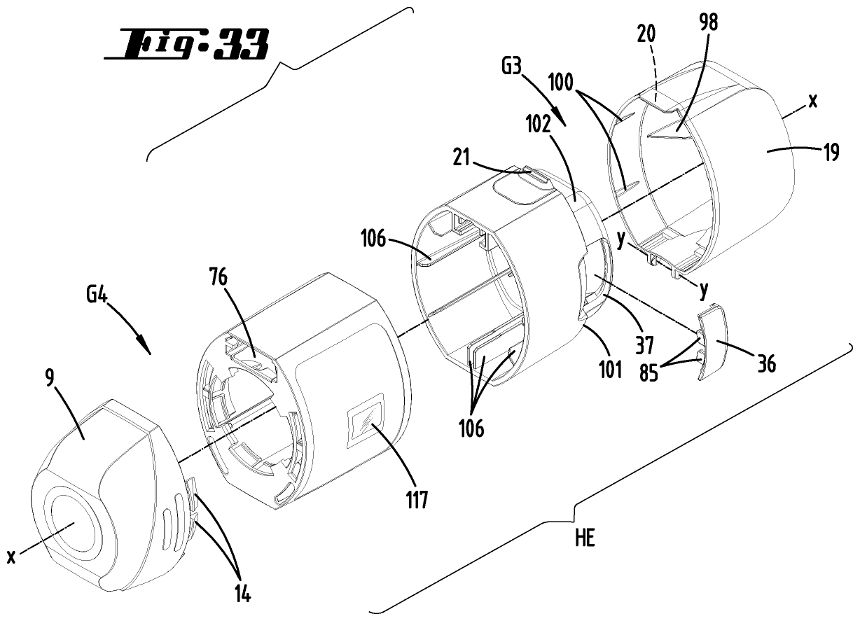

As can be seen, the functional elements FE form all parts relevant to the function as a nebulizer 1, whereby the functional elements FE shown in FIG. 3 are preferably part of all embodiments of a nebulizer 1 for single use according to FIGS. 1 to 29 and the functional elements FE in FIG. 32 are preferably part of all embodiments of a nebulizer 1 for multiple use according to FIGS. 30 to 44.

In principle, therefore, although only feasible in theory, inhalation can be carried out without the external handling elements HE, for example for testing and/or inspection purposes. This makes it possible, for example, to test the functional elements FE without concrete handling elements HE. A nebuliser torso consisting solely of the functional elements FE can advantageously be fitted with handling elements HE that differ, for example, in size and/or shape and/or colour design and/or choice of material.

Basically, the third (outer) housing part G3 is locked (e.g. latched) to the first (inner) housing part G1 in a rotationally fixed manner, just as the fourth (outer) housing part G4 is locked to the second (inner) housing part G2 in a rotationally fixed manner—as described above. The housing part G3 may also, as described above, in a different way be combined with the housing part G4, e.g. by welding, bonding or pressing.

The third housing part G3 may form a housing cover 101 extending substantially parallel to the parting plane E, the upwardly facing surface of which may provide a support surface for the cover cap 19 in the cap closed position.

A connection dome 102 with a central opening 103 can extend in the axial direction from and beyond the housing cover 101. In the area of this opening 103, radially inwardly pointing latching projections 104 are formed.

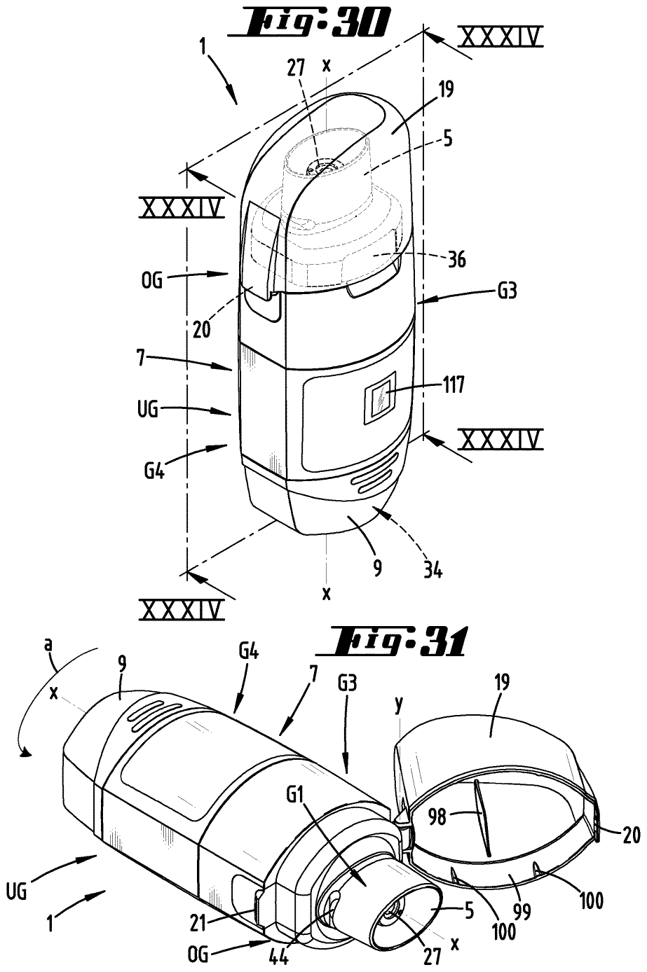

The latching projections 104 are designed and positioned accordingly for latching cooperation with the first housing part G1 in the region of a circumferential shoulder 105 or 105′, whereby axially differently positioned shoulders 105, 105′ can be used depending on the design and positioning of the latching projections 104. Thus, in a lip-like configuration of the connection dome 102 in a longitudinal section according to, for example, FIGS. 29 or 44, there can be a latching cooperation with a shoulder 105′ which, viewed in the axial direction, can be formed above a cooperation of the latching projections 104 with the axially offset shoulder 105 according to, for example, the further embodiments in FIGS. 8 and 24 or 34 and 39. The first housing part G1 can have differently positioned shoulders 105 corresponding to the optional arrangement of different third housing parts G3.

The interlocking of the housing parts G1 and G3 is given in an latching plane R offset upwards with respect to the parting plane E in the direction of the mouthpiece 5 with reference to the longitudinal sectional views. Furthermore, this latching plane R, irrespective of whether the shoulder 105 or the shoulder 105′ is used for latching, is also formed above a lower end 122 of the helical bevel surface 31, with a distance b (when the shoulder 105 is used) or b′ (when the shoulder 105′ is used) between the end 122 and the latching plane R, which distance can correspond, for example, to approximately 0.2 to 0.4 times the height c of the third housing part G3, which is also viewed in the axial direction.

In a further embodiment, the outer upper housing part G3 may have, on the inside of the wall and distributed over the circumference, a plurality of supporting ribs 106 which extend in the axial direction and, in the latching position, rest against a facing counter-surface of the inner housing part G1, for centring the third housing part G3 relative to the first housing part G1.

In addition, such supporting ribs 106 in cooperation with counter ribs 107 of the inner housing part G2 can effect the required rotationally fixed connection of the housing parts G1 and G3 (compare for example FIG. 7). Alternatively or in combination, this rotationally fixed connection can also be achieved by engagement of the above-described latching projections 104 in engagement pockets 108 of the inner housing part G1, which are provided individually over the circumference (compare, for example, FIG. 10).

To increase the dimensional stability, for example, two diametrically opposed stabilising ribs 109 can be provided on the upper outer housing part G3, each of which crosses the parting plane E and which, at least in the non-rotated basic position of the housing part pairs OG and UG relative to each other, can lie against the inside of the wall of the lower outer housing part G4. For this purpose, this contact area 110 can form a bead-like wall recess on the inside of the wall of the fourth housing part G4, into which the associated stabilising rib 109 falls in the basic position (compare, for example, FIGS. 15 and 18).

The lower fourth housing part G4 can, as shown in the embodiment examples of FIGS. 30 to 44, be of one-piece and pot- or sleeve-like design. Alternatively, however, a two-part design of the fourth housing part G4 is also possible according to the embodiment examples in FIGS. 1 to 29.

For example, two partial housing parts 111 and 112 can be provided, arranged one inside the other like a cup, which may be formed from different materials and/or in different colours and/or with different surface structures.

The partial housing parts 111 and 112 form an outer and an inner partial housing part, which are designed to be inserted into one another in alignment with the longitudinal axis x of the housing, whereby one partial housing part 111 can have projections 113 which engage in grooves 114 of the other partial housing part 112 for retention. In the retaining position, a wedging outwards results due to the corresponding cross-sectional design of the projections 113 and grooves 114 (compare, for example, FIG. 20).

In the outer partial housing part 111, the wall can be interrupted to form an aperture area 115. This is limited in the circumferential direction and towards a housing base. However, the aperture area 115 can open towards the parting plane E.

A filling section 116 of the inner partial housing part 112, which is thickened on the wall side or offset outwards, can engage in the aperture area 115, so that overall both partial housing parts 111 and 112 can each partially form a region of the, possibly circumferentially substantially step-free, outer surface of the fourth housing part G4 as a whole.

As can be seen from the illustrations in FIGS. 25 to 29 relating to the third embodiment, such aperture areas 115 and assignable filling sections 116 can also be provided, if necessary additionally, assigned to the housing base. Such filling sections 116 can also be formed as window parts 117, so that information lying behind them can be read through these window parts 117 exposed in the aperture area 115. Such information can be counter values of the counter 6 or also the content of a label or the like arranged on the container 2.

In the embodiments with replaceable container 2 and one-piece fourth housing part G4, a window part 117 may be recessed directly in the wall of housing part G4, in particular for reading the information of counter 6.

The lower housing part G4 can also have support ribs 123 formed on the inside of the wall for stabilisation and/or centring when attached to the second housing part G2.

In a nebulizer 1 for preferred single use, as shown in FIGS. 1 to 20, the outer housing parts G3 and G4 can be substantially oval and relatively uniformly rounded with respect to a plan view in which the longitudinal housing axis x is represented as a point. The outer, circumferential wall surface of the two housing parts G3 and G4 is preferably—with the exception of the hinge joint 8—not protruded radially by any housing element in such a plan view.

A nebulizer 1 for single use according to the second embodiment (FIGS. 21 to 24) can also have an essentially elongated rectangular shape in plan, with rounded corner areas 120 and convex broad sides 121 (compare in particular FIG. 21). In addition to the hinge joint 8, as also shown, the section of the cover cap 19 carrying the latching projection 14 can project substantially radially beyond the rest of the housing contour in the plan view with an overall nose-like design.

According to FIG. 25, a nebulizer 1 for single use can also have a ground plan which essentially starts from an octagon, with four broad sides 121 of spherical shape and four corner areas 120 which connect these broad sides 121 and which may be flattened. A window part 117 can be provided in one of the corner areas, through which, for example, information from the counter 6 can be read. In this embodiment, the trigger button 36 cannot be directly connected to the trigger ring 35 or to the retaining base 83. Instead, the trigger button 36 can be fastened to the upper outer housing part G3 via latching projections 85. The retaining base 83 preferably rests directly on the inside of the trigger button 36, which has a much larger surface area than the retaining base 83, so that pressure applied to the surface of the trigger button 36 from the outside results in the desired disengagement of the trigger ring 35.

As can be seen, different outer housing parts can also be provided for a nebulizer 1 with replaceable container 2. For example, the housing parts G3 and G4 of the fourth and fifth embodiments (FIGS. 30 to 34 and 35 to 39) are substantially based, at least in terms of external shape, on the second embodiment for disposable nebulizers shown in FIGS. 21 to 24.

The fourth embodiment according to FIGS. 30 to 34 is provided with a merely plug-in closure cap 9, wherein a window part 117 is arranged in a broad side 121 of the lower outer housing part G4, through which the counter 6 can be viewed.

In the fifth embodiment according to FIGS. 35 to 39, the closure cap 9 can only be pulled off downwards by actuating the key projection 78 after the latch has been released. Furthermore, in this embodiment a window part 117 for reading the counter information is provided in the area of a narrow side of the housing part G4, in which the key projection 78 also protrudes freely for actuation and to which narrow side the hinge joint 8 is also assigned.

FIGS. 40 to 44 show an embodiment of a nebulizer 1 for multiple use, which is based on the third embodiment with regard to its outer contour, correspondingly having a rounded octagonal ground plan. Here too, for example, both the window part 117 and the key projection 78 and the hinge joint 8 are assigned to a common narrow side of the housing 7 as a whole.

Part of the counter 6 of a nebulizer 1 for single use according to the embodiments shown in FIGS. 1 to 29 is first of all a spindle-like transmission part 45 with a geometric spindle axis z, which transmission part 45, when aligned parallel to the longitudinal housing axis x, has a thread 46 rising circumferentially on the outside along the spindle axis z. The spindle axis z is the geometric axis of the transmission part 45.

The transmission part 45 is freely rotatable about its spindle axis z in a bead-like recess 47 of the housing wall of the second housing part G2, whereby the transmission part 45 is further substantially rotatably and captively held at each end on the second housing part G2.

Along the shaft axis z, a counter pointer 48 meshing with the thread 46 of the transmission part 45 can be moved. Starting from a position in which the counter pointer 48 is assigned to the end of the second housing part G2 facing away from the mouthpiece 5, this pointer moves successively in the direction of the end region of the transmission part 45 facing the mouthpiece 5. Accordingly, with reference to the illustration in FIG. 8, for example, there is a successive movement of the counter pointer 48 from bottom to top.

The counter pointer 48 can point with its pointer tip directed transversely to the spindle axis z to a scale 49 arranged on the outside of the wall of the second housing part G2. The value to which the pointer tip of the counter pointer 48 points can, as preferred, indicate the number of possible atomisation processes still available or, alternatively, the number of atomisation processes already carried out.

This count value of the scale 49 is preferably visible to the user through a transparent section, for example through a window part 117, of the housing 7.

The transmission part 45 is rotationally driven by the relative rotational displacement of the housing part pairs OG and UG with respect to each other, which is carried out in the course of the inhalation preparation, for which purpose the transmission part 45 has a tooth formation 50 in the region of the end facing the mouthpiece 5, with, for example, four radially aligned teeth evenly distributed in the circumferential direction.

On the inside of the wall of the first (upper) housing part G1, in the area of a free end facing away from the mouthpiece 5 and further essentially in the plane of the tooth formation 50, two diametrically opposed drivers 51, which are approximately U-shaped in a plane transverse to the longitudinal axis x of the housing, are formed for cooperation with the tooth formation 50 when the pairs of housing part pairs OG and UG are rotated relative to each other. Due to the diametrical arrangement of two drivers 51, a rotational effect on the transmission part 45 and thus a linear displacement on the counter pointer 48 along the spindle axis x is achieved with each inhalation preparation.

When a maximum number of nebulisation processes has been reached, i.e. when a predetermined maximum use of the nebuliser 1, which is preferably designed for single use, has been reached, the nebuliser 1 should no longer be usable, since in this situation it cannot be ensured that the container 2 still contains a sufficient amount of fluid to carry out proper inhalation. A supposed inhalation without or with reduced fluid output is to be prevented.

For this purpose, the nebulizer 1 has a locking part 52 which, in the locked position, prevents the axial displaceability of the retaining part 16 for building up the tension of the compression spring 28 and for filling the pressure chamber 4.

The locking part 52 is preferably made of a stamped and bent spring plate.

Further preferably, the locking part 52 is held on the outside of the wall of the second housing part G2. For this purpose, a radially outwardly open pocket 54 can be provided in the housing wall substantially as an extension of the bead-like recess 47 for receiving the spindle-shaped transmission part 45, in which pocket 54 the locking part 52 is axially displaceable and captive to a limited extent.

In a release position, in which the aforementioned maximum number of atomisation processes has not yet been reached, the locking part 52 is locked in the pocket 54.

To assume the locking position, the counter pointer 48 of the counter 6, which moves in the direction of the locking part 52 in the course of the atomisation processes, is firmly connected to a finger-like release part 55. This release part 55 moves essentially in the direction of the spindle axis z and has a control finger at its end facing the mouthpiece 5.

When the maximum number of atomisation processes is reached, the release part 55, which is displaced together with the counter pointer 48, reaches a contact surface of the locking part 52 with its control finger and lifts this in such a way that a preferred locking is cancelled. In the course of the further displacement of the release part 55, the locking part 52 can now be displaced in the axial direction to such an extent that ultimately a bend 53 of the locking part 52 loses its support on the bottom of the pocket and falls through an opening 56 formed in the bottom of the pocket 54 with spring support, in order to engage through this in an axial groove 57 of the retaining part 16, which is open radially outwards in the direction of the inner surface of the second housing part G2 overlapping the retaining part 16.

According to this, the retaining part 16 as a whole, correspondingly together with the container 2, is prevented from an axial movement, preferably permanently, in the stop-limited basic position. The nebulizer 1 according to the first, second and third embodiments cannot be used any further in this locked position which cannot be undone by the user, preferably without being destroyed.

Part of the counter 6 of a multiple use nebulizer 1, as shown for example in FIGS. 30 to 44 relating to a fourth, fifth and sixth embodiment, is a transmission part 58 which transmits the relative rotational displacement of the housing part pairs UG and OG relative to each other to a sleeve part 59 of the counter 6. This sleeve part 59 is provided with characters 60 applied to an outer surface, which represent the counts.

The transmission part 58 is designed in the manner of a transmission shaft, with an axis of rotation z which runs in the same direction as the longitudinal axis x of the housing. The transmission shaft 58 is further designed and arranged in such a way that it interacts directly both with the upper housing part pair OG, in particular with the upper inner housing part G1 enclosed by the housing part G3, and with the lower housing part pair UG, in particular with the lower inner housing part G2, as well as further directly with the sleeve part 59 of the counter 6.

For this purpose, the transmission part 58 can be rotatably held in a niche 61 formed on the outside of the wall of the second housing part G2 and open radially outwards, wherein both the niche 61 and the transmission part 58 in the position of use pass through a transverse plane extending transversely to the longitudinal axis x of the housing, in which transverse plane a collar-like circumferential first support flange 62 is simultaneously formed on the outside of the wall of the second housing part G2.

Associated with the niche 61, the first support flange 62 is provided with an open-edge passage opening 63.

The transmission part 58 has two areas spaced apart from each other in the direction of their axis of rotation z, with a first toothed formation 64 and a second toothed formation 65.

The first toothed formation 64 may be formed by four counting fingers evenly distributed in the circumferential direction on the transmission shaft.

The first housing part G1, which is connected to the third housing part G3 in a rotationally fixed manner, encloses the second housing part G2 in a region of the first toothed formation 64 with a free wall end section pointing in the direction of the sleeve part 59. A driver 66, which is approximately U-shaped in a plane transverse to the longitudinal axis x of the housing, is integrally formed on the inner wall side of this wall end section, correspondingly facing the second housing part G2, for cooperation with the first toothed formation 64.

With reference to the illustrations, a first toothing of the sleeve part 59 cooperating with the second toothed formation 65 is formed on the inside of the sleeve in an upper edge region of the sleeve part 59. At an axial distance therefrom in the region of a lower edge, a second circumferential toothing 67 is preferably also formed on the inside of the sleeve for cooperation with a spring-loaded non-return finger formed on the second housing part G2.

With a preferred number of 30 to 35 teeth, further preferably 32 teeth, of the first toothing, a toothed area extending in the circumferential direction over a total angle of about 330 to 340 degrees, further preferably about 336 degrees, can result. Over the remaining circumferential portion of, for example, 20 to 30 degrees, further for example 24 degrees, there is a tooth gap which provides a freewheel for the driving finger of the transmission part 58, particularly in the course of a container change described in more detail below.

The characters 60 applied to the outer surface are visible to the user from the outside through a transparent viewing window, for example window part 117, provided in the fourth housing part G4, in particular the character 60 representing the current filling status of the inserted container 2.

With a rotation of the lower pair of housing parts UG in the direction of rotation a relative to the fixed upper housing part pair OG in preparation for an atomisation process, a rotation of the transmission part 58 as a whole about its axis of rotation z by preferably 180 degrees can be effected due to the action of the driver 66 provided in the upper inner housing part G1 on the first toothed formation 64 of the transmission part 58.

In conjunction with the driving finger, this results in a rotation of the second toothed formation 65 with the driving finger by 180 degrees every second time an atomisation preparation position is reached, according to the action on the transmission part 58 via the driver 66. The attainment of every fourth preparatory spraying position causes a rotational effect on the sleeve part 59 via the driving finger, which is then rotated through a total of 360 degrees. The driving finger moves the sleeve part 59 further by one tooth, correspondingly preferably the sleeve part 59 through an angle of 10.5 degrees.

In an advantageous manner, the counter 6 and, via this, the nebulizer 1 designed for multiple use is blocked by a blocking device in the sense of the rotatability of the housing part pairs OG and UG relative to each other, in particular when a minimum count value (for example zero) is reached. Accordingly, the blockage does not allow further rotation of the lower housing part pair UG relative to the upper housing part pair OG. The user is thus also made aware haptically of an empty container 2 without having to look separately at the counter display. The nebulizer 1 can no longer be used without further measures.

For this purpose, the locking device preferably has a locking pawl 77 with a locking projection 68. The locking pawl 77 can be held in a guide 69 formed on the inside of the wall of the first housing part G1 so as to be displaceable in the direction of the longitudinal axis x of the housing, preferably with a limited number of stops. In this case, the locking pawl 77 is further preferably spring-loaded via a spring 70, for example, as also shown, via a cylinder compression spring, in the direction of a detent position according to the illustrations downwards, in the direction of the sleeve part 59.

The spring 70 is supported on the inside of the wall of the outer third housing part G3 on a correspondingly formed base 73.

In normal operation of the nebulizer 1, the locking pawl 77 is supported with its locking projection 68 on the tip side on a second support flange 71 surrounding the first toothing of the sleeve part 59 or—possibly depending on the rotational position—on the first support flange 62 of the lower inner housing part G2. The support surface of the second support flange 71 extends transversely to the longitudinal axis x of the housing, preferably viewed along the longitudinal axis x of the housing, essentially directly below the first support flange 62, offset in the plane in the direction of the base part 29.

Viewed over the circumference, this second support flange 71 is also partially interrupted. Accordingly, there is a circumferential locking opening 72 of the sleeve part 59 in this area, into which locking opening 72, in particular when the minimum count value (for example zero) is reached, the locking projection 68 automatically retracts in a locking manner due to the spring load described above.

In such a count-minimum position of the sleeve part 59, the passage opening 63 of the first supporting flange 62 formed on the lower inner housing part G2 and the locking opening 72 of the second support flange 71 formed on the sleeve part 59 are in overlap—with respect to the direction of extension of the longitudinal axis x of the housing. Furthermore, both flange openings (passage opening 63 and locking opening 72) extend together in the direction of the travel of the locking pawl 77. In this circumferential alignment of the flange openings with the locking projection 68, the locking pawl 77, spring-loaded by the latter, can enter a locking position in which the locking pawl 77 crosses the transverse plane given by the supporting flanges and, by bearing in the locking opening 72 against a locking shoulder preferably extending perpendicularly to the second support flange 71, effects a rotational locking between the housing part pairs OG and UG in the usual rotational direction a.

In the illustrated embodiment examples according to FIGS. 30 to 44, this locking of the counter 6 can, as is also preferred, only be released by changing the container 2. In particular, the container connection part 13, which is connected to the container 2 and can preferably only be separated from the container 2 by destruction, serves this purpose.

The container connection part 13 can, as is also preferred, be a plastic injection-moulded part, further in particular having a pot-like bottom section 74 with a pot-shaped recess in which the container 2 is received at the foot end, i.e. in the region of the end opposite the latching groove 18. An cantilever 75 is formed on this bottom section 74, which is aligned in the direction of the longitudinal axis x of the housing in the associated state and extends adjacent to the container 2 in the direction of the mouthpiece 5. When a container 2 is inserted into the housing 7 with the closure cap 9 removed, this cantilever 75 can be inserted into a guide receptacle 76 of the housing 7, in particular of the lower outer housing part G4. The guide receptacle 76 runs adjacent to the receiving room 10 for the container 2.

An insertion locking part is preferably arranged in the cantilever 75. In addition, the cantilever 75 facing the container 2 can have a control projection.

With the insertion of an unused container 2 with its container connection part 13 into the housing 7, the cantilever 75 is inserted into the guide receptacle 76. As a result of sliding over the cantilever 75 in the course of the further insertion process, for example on a helical bevel surface, the sleeve part 59 is forced to rotate to its home position. The home position is preferably the count position in which the maximum count value is displayed (e.g. 120, 60 or 30).

As a result of the formation of the tooth gap described in the area of the first toothing of the sleeve part 59, there is a free run for the driving finger, so that the adjustment of the sleeve part 59 into the home position is made possible without any possible cancellation of a toothing engagement, for example by lifting or displacing a toothing engagement part, which is carried out specifically for this purpose alone.

The angle of rotation achieved in this process essentially corresponds to the angular dimension over which the tooth gap of the sleeve part 59 extends.

In the course of the thus achieved rotation of the sleeve part 59 in the course of the insertion process of a preferably new, unused container 2, the locking pawl 77 with its locking projection 68 is lifted out of the blocking position against the force of the spring 70. The locking projection 68 is then supported by the spring on the facing surface of the second support flange 71 in the basic or initial position that has then been reached.

From this basic position, during a usual rotation of the lower housing part pair UG against the upper housing part pair OG, the locking projection 68 can be lifted onto the first support flange 62 and slide on it.

Preferably, only the insertion of a non-pre-used container 2 is provided. On the other hand, a pre-used container 2, including an emptied container 2, is prevented from being (re)inserted into the housing 7. This is done by the mentioned insertion locking part of the container connection part 13, which after a first insertion of the container 2 into the housing 7 or with the removal of the container 2 from the housing 7 falls into a blocking position, which does not allow a renewed insertion of this container 2.

The insertion locking part may, for example, be pivotally mounted on the cantilever 75 with a related geometric pivot axis which may extend transversely to the longitudinal axis x of the housing.

The foregoing serves to explain the inventions covered by the application as a whole, which also independently advance the prior art at least by means of the following combinations of features in each case, it being possible for two, more or all of these combinations of features also to be combined, namely:

A nebulizer 1 characterised in that a third housing part G3 is provided, which surrounds the first housing part G1 on the outside and is connected to the first housing part G1 in a rotationally fixed manner.

A nebulizer 1 characterised in that the third housing part G3 is engaged with the first housing part G1 above a lower end 122 of the helical bevel surface 31.

A nebulizer 1 characterised in that the first housing part G1 forms a circumferential shoulder 105, 105′ for a latching stop when the third housing part G3 is placed on the first housing part G1.

A nebulizer 1 characterised in that a fourth housing part G4 is snap-fitted to the second housing part G2.

A nebulizer 1 characterised in that the fourth housing part G4 lies opposite the third housing part G3 in a parting plane E extending transversely to the longitudinal axis x of the housing, in which parting plane E the third and fourth housing parts G3 and G4 are rotatable relative to one another.

A nebulizer 1 characterised in that a cover cap 19 is attached to the third housing part G3.

A nebulizer 1 characterised in that the cover cap 19 is connected to the third housing part G3 via a hinge joint 8.

A nebulizer 1 characterised in that the fourth housing part G4 consists of two partial housing parts 111, 112 arranged one inside the other like a cup.

A Nebulizer 1 characterized in that the partial housing parts 111, 112 of the fourth housing part G4 have grooves 114 formed in the direction of the longitudinal housing axis x and projections 113 for bringing the partial housing parts 111, 112 together in the vertical direction.

A nebulizer 1 characterised in that the outer partial housing part 111 and the inner partial housing part 112 each partially form a portion of the outer surface of the fourth housing part G4.

A nebulizer 1 characterised in that the inner partial housing part 112 has a window part 117 exposed in an aperture area 115 of the outer partial housing part 111.

A nebulizer 1 characterised in that a locking pawl 77 is guided on the first housing part G1 in a manner fixed to the direction of rotation and, in a selected position of a sleeve part 59 surrounding the second housing part G2, enters a locking opening 72 of the sleeve part 59 by displacement in the direction of the longitudinal axis x of the housing, and in that the locking pawl 77 is supported in a biased manner on the third housing part G3.

All disclosed features are essential to the invention (individually, but also in combination with each other). The disclosure of the application hereby also includes the full disclosure content of the associated/attached priority documents (copy of the previous application), also for the purpose of including features of these documents in the claims of the present application. The subclaims, even without the features of a referenced claim, characterise with their features independent inventive further developments of the prior art, in particular in order to file divisional applications on the basis of these claims. The invention indicated in each claim may additionally have one or more of the features indicated in the above description, in particular those provided with reference numerals and/or in the list of reference numerals. The invention also relates to forms of design in which individual features mentioned in the above description are not realised, in particular to the extent that they are recognisably dispensable for the respective intended use or can be replaced by other means having the same technical effect.

### REFERENCE LIST

1 Nebulizer 30 Opening
2 Container 31 Helical bevel surface
3 Fluid 32 Counter bevel surface
4 Pressure chamber 33 Piercing needle
5 Mouthpiece 34 Spring plate member
6 Counter 35 Trigger ring
7 Housing 36 Trigger button
8 Hinged joint 37 Window
9 Closure cap 38 Wall
10 Receiving room 39 Receptacle
11 Container wall 40 Control projection
12 Container bottom 41 Counter control projection
13 Container connection part 42 Recess
14 Latching projection 43 Aerosol
15 Latching shoulder 44 Ventilation opening
16 Retaining part 45 Transmission part
17 Latching projection 46 Thread
18 Latching groove 47 Recess
19 Cover cap 48 Counter pointer
20 Latching section 49 Scale
21 Counter latching section 50 Tooth formation
22 Holder 51 Driver
23 Suction pipe 52 Locking part
24 Passage opening 53 Bend
25 Non-return valve 54 Pocket
26 Nozzle body 55 Release part
27 Ejection nozzle 56 Opening
28 Compression spring 57 Axial groove
29 Base part 58 Transmission part
59 Sleeve part 89 Base section
60 Characters 90 Hinge bracket
61 Niche 91 Hinge eye
62 First support flange 92 Recess
63 Passage opening 93 Latching bead
64 First toothed formation 94 Hinge arm
65 Second toothed formation 95 Hinge pin
66 Driver 96 Overflow slope
67 Second toothing 97 Counter bead
68 Locking projection 98 Support rib
69 Guide 99 Cap section
70 Spring 100 Projection
71 Second support flange 101 Housing cover
72 Locking opening 102 Connection dome
73 Base 103 Opening
74 Bottom section 104 Latching projection
75 Cantilever 105 Shoulder
76 Guide receptacle 105′ Shoulder
77 Locking pawl 106 Supporting rib
78 Key projection 107 Counter rib
79 Slot opening 108 Pocket
80 Release lever 109 Stabilising rib
81 Spring arm 110 Contact area
82 Opening 111 Partial housing part
83 Retaining base 112 Partial housing part
84 Contact surface 113 Projection
85 Latching projection 114 Groove
86 Latching receptacles 115 Aperture area
87 Support surface 116 Filling section
88 Latching rib 117 Window part
118 Latching projection FE Functional elements
119 Counter latching projection G1 first housing part
120 Corner area G2 second housing part
121 Broad side G3 third housing part
122 End G4 fourth housing part
123 Support rib HE Handling elements
OG upper housing part pair
R Latching plane
UG lower housing part pair
a Direction of rotation
b Distance
b′ Distance
C Height
u Axis
X Longitudinal housing axis
y Pivot axis
Z Spindle axis
E Parting plane

1. Nebulizer (1) for a fluid (3) located in a container (2), having a first housing part (G1), which has a mouthpiece (5), and a second housing part (G2), which forms a receiving room (10) for the container (2), the first and second housing parts (G1 and G2) being rotatable relative to one another about a longitudinal housing axis (x) in preparation for an atomising process, the first housing part (G1) further having a helical bevel surface (31), for acting on a retaining part (16) for the container (2), which retaining part (16) is axially displaceable relative to the first and second housing parts (G1 and G2) when connected rotationally fixed to the second housing part (G2), characterised in that a third housing part (G3) is provided, which surrounds the first housing part (G1) on the outside and is connected to the first housing part (G1) in a rotationally fixed manner.

2. Nebulizer according to claim 1, characterised in that the third housing part (G3) is latched to the first housing part (G1) above a lower end (122) of the helical bevel surface (31).

3. Nebulizer according to one of the preceding claims, characterised in that the first housing part (G1) forms a circumferential shoulder (105, 105′) for a latching stop when the third housing part (G3) is placed on the first housing part (G1).

4. Nebulizer according to one of the preceding claims, characterised in that a fourth housing part (G4) is fastened in a snap-in manner to the second housing part (G2).

5. Nebulizer according to one of the preceding claims, characterised in that the fourth housing part (G4) is opposite the third housing part (G3) in a parting plane (E) extending transversely to the longitudinal axis (x) of the housing, in which parting plane (E) the third and fourth housing parts (G3 and G4) are rotatable relative to one another.

6. Nebulizer according to one of the preceding claims, characterised in that a cover cap (19) is attached to the third housing part (G3).

7. Nebulizer according to one of the preceding claims, characterised in that the cover cap (19) is connected to the third housing part (G3) via a hinge joint (8).

8. Nebulizer according to one of the preceding claims, characterised in that the fourth housing part (G4) consists of two partial housing parts (111, 112) arranged one inside the other in a pot-like manner.

9. Nebulizer according to one of the preceding claims, characterised in that the partial housing parts (111, 112) of the fourth housing part (G4) have grooves (114) formed in the direction of the longitudinal axis (x) of the housing and projections (113) for bringing the partial housing parts (111, 112) together in the vertical direction.

10. Nebulizer according to any one of the preceding claims, characterised in that the outer partial housing part (111) and the inner partial housing part (112) each partially form a portion of the outer surface of the fourth housing part (G4).

11. Nebulizer according to any one of the preceding claims, characterised in that the inner partial housing part (112) has a window part (117) exposed in an aperture area (115) of the outer partial housing part (111).

12. Nebulizer according to one of the preceding claims, characterised in that a locking pawl (77) is guided on the first housing part (G1) in a manner fixed to the direction of rotation, which pawl, in a selected position of a sleeve part (59) surrounding the second housing part (G2), enters a locking opening (72) of the sleeve part (59) by displacement in the direction of the longitudinal axis (x) of the housing, and in that the locking pawl (77) is supported in a prestressed manner on the third housing part (G3).

13. Nebulizer characterised by one or more of the identifying features of any one of the preceding claims.
